(19) United States
(12) Patent Application Publication
(10) Pub. No.: US 2017/0079741 A1
(43) Pub. Date: Mar. 23, 2017

(54) SCANNING PROJECTION APPARATUS, PROJECTION METHOD, SURGERY SUPPORT SYSTEM, AND SCANNING APPARATUS

(71) Applicant: NIKON CORPORATION, Tokyo (JP)

(72) Inventor: Susumu MAKINOUCHI, Tokyo (JP)

(73) Assignee: NIKON CORPORATION, Tokyo (JP)

(21) Appl. No.: 15/367,333

(22) Filed: Dec. 2, 2016

Related U.S. Application Data
(63) Continuation of application No. PCT/JP2014/064989, filed on Jun. 5, 2014.

(51) Int. Cl. A61B 90/00; A61B 5/00; G01N 21/359

(52) U.S. Cl. CPC A61B 90/36; G01N 21/359; A61B 5/0071; A61B 5/4872; A61B 5/4875; A61B 5/4836; A61B 2090/366

(57) ABSTRACT

A scanning projection apparatus includes: an irradiation unit that irradiates a biological tissue with detection light; a light detection unit that detects light that is radiated from the tissue irradiated with the detection light; an image generation unit that generates data on an image about the tissue by using a detection result of the light detection unit; and a projection unit including a projection optical system that scans the tissue with visible light on the basis of the data, the projection unit being configured to project the image on the tissue through the scanning with the visible light.

### CROSS REFERENCE TO RELATED APPLICATION

This is a Continuation of PCT Application No. PCT/JP2014/064989, filed on Jun. 5, 2014. The contents of the above-mentioned application are incorporated herein by reference.

### FIELD OF THE INVENTION

The present invention relates to a scanning projection apparatus, a projection method, a surgery support system, and a scanning apparatus.

### BACKGROUND

In medical and other fields, a technology of projecting an image on a tissue is proposed (see, for example, Patent Literature 1). For example, the apparatus according to Patent Literature 1 irradiates a body tissue with infrared rays, and acquires an image of subcutaneous vessels on the basis of infrared rays reflected by the body tissue. This apparatus projects a visible light image of the subcutaneous vessels on the surface of the body tissue.

[Patent Literature 1] Japanese Unexamined Patent Application Publication No. 2006-102360

The surface of a tissue may be uneven, and it may be difficult to focus an image when the image is projected on the surface of the tissue. As a result, a projection image projected on the surface of the tissue may be unfocused, and the displayed image may be blurred. It is an object of the present invention to provide a scanning projection apparatus, a projection method, a surgery support system, and a scanning apparatus that are capable of projecting a sharp image on a biological tissue.

### SUMMARY

A first aspect of the present invention provides a scanning projection apparatus including: an irradiation unit that irradiates a biological tissue with detection light; a light detection unit that detects light that is radiated from the tissue irradiated with the detection light; an image generation unit that generates data on an image about the tissue by using a detection result of the light detection unit; and a projection unit including a projection optical system that scans the tissue with visible light on the basis of the data, the projection unit being configured to project the image on the tissue through the scanning with the visible light.

A second aspect of the present invention provides a projection method including: irradiating a biological tissue with detection light; detecting, by a light detection unit, light that is radiated from the tissue irradiated with the detection light; generating data on an image about the tissue by using a detection result of the light detection unit; and scanning the tissue with visible light on the basis of the data, and projecting the image on the tissue through the scanning with the visible light.

A third aspect of the present invention provides a surgery support system including: the scanning projection apparatus in the first aspect; and an operation device that is capable of treating the tissue in a state in which the image is projected on the tissue by the scanning projection apparatus.

A fourth aspect of the present invention provides a surgery support system including: the scanning projection apparatus in the first aspect.

A fifth aspect of the present invention provides scanning apparatus including: an irradiation unit that irradiates a target with detection light; a detection unit that detects light that is radiated from the target irradiated with the detection light; a generation unit that generates data on water or lipid in the target on the basis of a detection result of the detection unit; and a scanning unit that scans the target with visible light on the basis of the data on water or lipid.

A sixth aspect of the present invention provides a surgery support system including: the scanning projection apparatus in the fifth aspect.

According to the present invention, a scanning projection apparatus, a projection method, a surgery support system, a scanning apparatus that are capable of projecting a sharp image on a biological tissue can be provided.

### BRIEF DESCRIPTION OF DRAWINGS

FIG. 1 is a diagram showing a scanning projection apparatus according to a first embodiment.

FIG. 2 is a conceptual diagram showing an example of pixel arrangement of an image according to the present embodiment.

FIG. 3 is a graph showing a distribution of absorbance in a near-infrared wavelength region according to the present embodiment.

FIG. 4 is a flowchart showing a projection method according to the first embodiment.

FIG. 5 is a diagram showing a modification of an irradiation unit according to the present embodiment.

FIG. 6 is a diagram showing a modification of a light detection unit according to the present embodiment.

FIG. 7 is a diagram showing a modification of a projection unit according to the present embodiment.

FIG. 8 is a diagram showing a scanning projection apparatus according to a second embodiment.

FIG. 9 is a timing chart showing an example of operation of an irradiation unit and a projection unit according to the present embodiment.

FIG. 10 is a diagram showing a scanning projection apparatus according to a third embodiment.

FIG. 11 is a diagram showing a scanning projection apparatus according to a fourth embodiment.

FIG. 12 is a diagram showing an example of a surgery support system according to the present embodiment.

FIG. 13 is a diagram showing another example of the surgery support system according to the present embodiment.

### DETAILED DESCRIPTION OF EMBODIMENTS

### First Embodiment

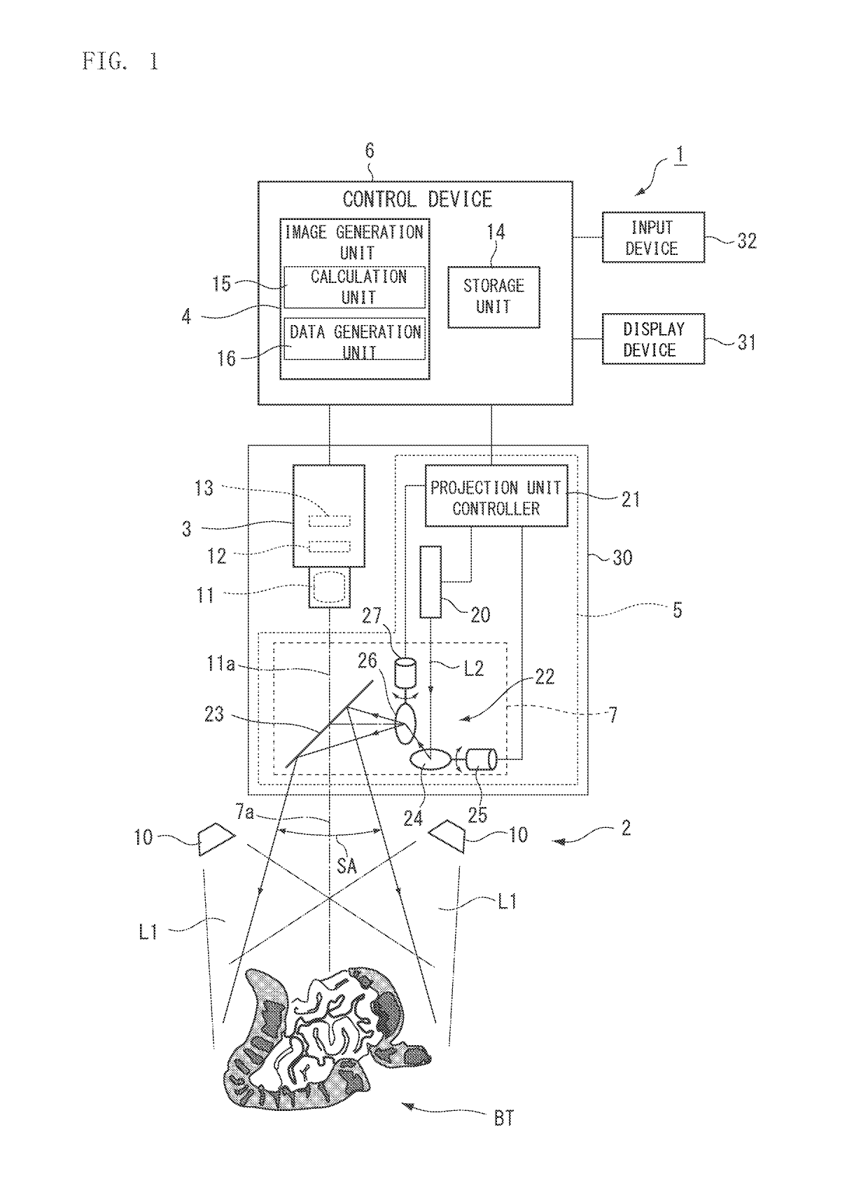

A first embodiment will be described. FIG. 1 is a diagram showing a scanning projection apparatus 1 according to the present embodiment. The scanning projection apparatus 1 detects light radiated from a biological (for example, animal) tissue BT, and projects an image about the tissue BT on the tissue BT by using the detection result. The scanning projection apparatus 1 can display an image including information on the tissue BT directly on the tissue BT. Examples of the light radiated from the biological tissue BT include light (for example, infrared light) obtained by irradiating the tissue BT with infrared light, and fluorescent light that is emitted when a tissue BT labeled with a light emitting substance such as fluorescent dye is irradiated with excitation light.

The scanning projection apparatus 1 can be used for a surgical operation, such as a laparotomy. The scanning projection apparatus 1 projects information on an affected area analyzed with use of near-infrared light directly on the affected area. The scanning projection apparatus 1 can display an image indicating components of the tissue BT as an image about the tissue BT. The scanning projection apparatus 1 can display an image in which a particular component in the tissue BT is emphasized as an image about the tissue BT. Examples of such an image include an image indicating the distribution of lipid in the tissue BT and an image indicating the distribution of water in the tissue BT. For example, the image can be used to determine the presence/absence of tumor in an affected area (tissue BT). The scanning projection apparatus 1 can superimpose an image about an affected area on the affected area for display. An operator can perform a surgery while directly viewing information displayed in the affected area. The operator or another person (such as a support person or a health professional) may operate the scanning projection apparatus 1.

The scanning projection apparatus 1 can be applied to invasive procedures involving an incision of a tissue BT, such as a general operation, as well as various kinds of non-invasive procedures involving no incision of a tissue BT, such as medical applications, test applications, and examination applications. For example, the scanning projection apparatus 1 can be used also for clinical examinations, such as blood sampling, pathological anatomy, pathological diagnosis, and biopsy. A tissue BT may be a tissue of a human or may be a tissue of a living organism other than a human. For example, the tissue BT may be a tissue cut away from a living organism, or may be a tissue attached to a living organism. For example, the tissue BT may be a tissue (biological tissue) of a living organism (living body) or may be a tissue of a dead organism (dead body). The tissue BT may be an object excised from a living organism. For example, the tissue BT may include any organ of a living organism, may include a skin, and may include a viscus, which is on the inner side of the skin.

The scanning projection apparatus 1 includes an irradiation unit 2, a light detection unit 3, an image generation unit 4, and a projection unit 5. In the present embodiment, the scanning projection apparatus 1 includes a control device 6 that controls each unit in the scanning projection apparatus 1, and the image generation unit 4 is provided in the control device 6.

Each unit in the scanning projection apparatus 1 schematically operates as follows. The irradiation unit 2 irradiates a biological tissue BT with detection light L1. The light detection unit 3 detects light that is radiated from the tissue BT irradiated with the detection light L1. The image generation unit 4 generates data on an image about the tissue BT by using the detection result of the light detection unit 3. The projection unit 5 includes a projection optical system 7 that scans the tissue BT with visible light L2 on the basis of the data. The projection unit 5 projects an image (projection image) on the tissue BT through the scanning with the visible light L2. For example, the image generation unit 4 generates the projection image by arithmetically processing the detection result of the light detection unit 3.

Next, each unit in the scanning projection apparatus 1 will be described. In the present embodiment, the irradiation unit 2 includes a light source 10 that emits infrared light. The light source 10 includes, for example, an infrared light emitting diode (infrared LED), and emits infrared light as the detection light L1. The light source 10 emits infrared light having a wider wavelength band than that of a laser light source. The light source 10 emits infrared light in a wavelength band including a first wavelength, a second wavelength, and a third wavelength. As described in detail later, the first wavelength, the second wavelength, and the third wavelength are wavelengths used to calculate information on particular components in the tissue BT. The light source 10 may include a solid-state light source other than an LED, or may include a lamp light source such as a halogen lamp.

The light source 10 is fixed so that, for example, a region to be irradiated with detection light (detection light irradiation region) is not moved. The tissue BT is disposed in the detection light irradiation region. For example, the light source 10 and the tissue BT are disposed such that relative positions thereof are not changed. In the present embodiment, the light source 10 is supported independently from the light detection unit 3 and supported independently from the projection unit 5. The light source 10 may be fixed integrally with at least one of the light detection unit and the projection unit 5.

The light detection unit 3 detects light that travels via the tissue BT. The light that travels via the tissue BT includes at least a part of the light reflected by the tissue BT, light transmitted through the tissue BT, and light scattered by the tissue BT. In the present embodiment, the light detection unit 3 detects infrared light reflected and scattered by the tissue BT.

In the present embodiment, the light detection unit 3 detects infrared light having the first wavelength, infrared light having the second wavelength, and infrared light having the third wavelength separately. The light detection unit 3 includes an imaging optical system 11, an infrared filter 12, and an image sensor 13.

The imaging optical system 11 includes one or two or more optical elements (for example, lenses), and is capable of forming an image of the tissue BT irradiated with the detection light L1. The infrared filter 12 transmits infrared light having a predetermined wavelength band among light passing through the imaging optical system 11, and blocks infrared light in wavelength bands other than the predetermined wavelength band. The image sensor 13 detects at least a part of the infrared light radiated from the tissue BT via the imaging optical system 11 and the infrared filter 12.

The image sensor 13 includes a plurality of light receiving elements arranged two-dimensionally, such as a CMOS sensor or a CCD sensor. The light receiving elements are sometimes called pixels or subpixels. The image sensor includes photodiodes, a readout circuit, an A/D converter, and other components. The photodiode is a photoelectric conversion element that is provided for each light receiving element and generates electric charges by infrared light entering the light receiving element. The readout circuit reads out the electric charges accumulated in the photodiode for each light receiving element, and outputs an analog signal indicating the amount of electric charges. The A/D converter converts the analog signal read out by the readout circuit into a digital signal.

In the present embodiment, the infrared filter 12 includes a first filter, a second filter, and a third filter. The first filter, the second filter, and the third filter transmit infrared light having different wavelengths. The first filter transmits infrared light having a first wavelength and blocks infrared light having a second wavelength and a third wavelength. The second filter transmits infrared light having the second wavelength and blocks infrared light having the first wavelength and the third wavelength. The third filter transmits infrared light having the third wavelength and blocks infrared light having the first wavelength and the second wavelength.

The first filter, the second filter, and the third filter are disposed correspondingly to the arrangement of the light receiving elements so that infrared light entering each light receiving element may be transmitted through any one of the first filter, the second filter, and the third filter. For example, infrared light having the first wavelength transmitted through the first filter enters a first light receiving element of the image sensor 13. Infrared light having the second wavelength transmitted through the second filter enters a second light receiving element adjacent to the first light receiving element. Infrared light having the third wavelength transmitted through the third filter enters a third light receiving element adjacent to the second light receiving element. In this manner, the image sensor 13 uses three adjacent light receiving elements to detect the light intensity of infrared light having the first wavelength, infrared light having the second wavelength, and infrared light having the third wavelength radiated from a part on the tissue BT.

In the present embodiment, the light detection unit 3 outputs the detection result of the image sensor 13 as a digital signal in an image format (hereinafter referred to as captured image data). In the following description, an image captured by the image sensor 13 is referred to as captured image as appropriate. Data on the captured image is referred to as captured image data. The captured image is of a full-spec high definition (HD format) for the sake of description, but there are no limitations on the number of pixels of the captured image, pixel arrangement (aspect ratio), the gray-scale of pixel values, and the like.

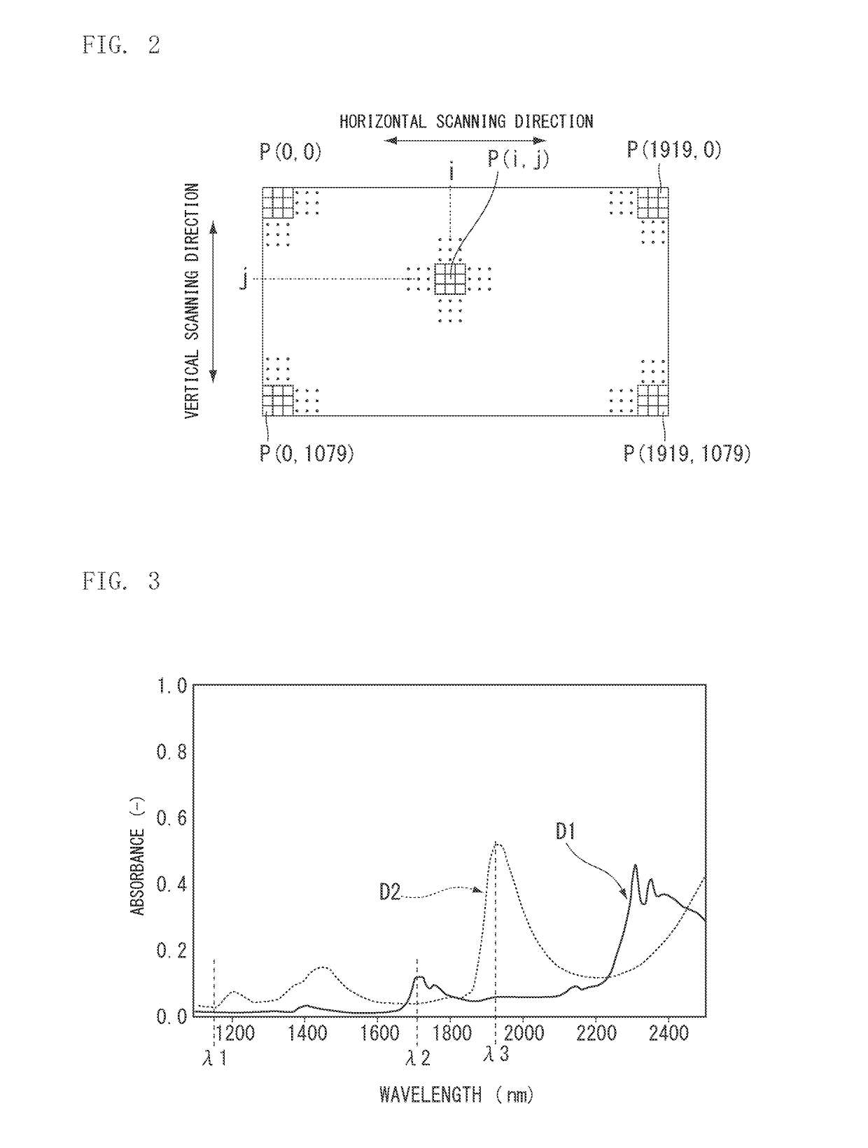

FIG. 2 is a conceptual diagram showing an example of pixel arrangement of an image. In an HD-format image, 1920 pixels are arranged in a horizontal scanning line direction, and 1080 pixels are arranged in a vertical scanning line direction. The pixels arranged in line in the horizontal scanning line direction are sometimes called horizontal scanning line. The pixel value of each pixel is represented by data of 8 bits, for example, and is represented by 256 gray scales of 0 to 255 in decimal notation.

As described above, the wavelength of infrared light detected by each light receiving element of the image sensor 13 is determined by the position of the light receiving element, and hence each pixel value of captured image data is associated with the wavelength of infrared light detected by the image sensor 13. The position of a pixel on the captured image data is represented by (i,j), and the pixel disposed at (i,j) is represented by P(i,j). i is the number of a pixel and is incremented in ascending order of 1, 2, 3, starting from a pixel at one edge in the horizontal scanning direction as 0, toward the other edge. j is the number of a pixel and is incremented in ascending order of 1, 2, 3, starting from a pixel at one edge in the vertical scanning direction as 0, toward the other edge. In an HD-format image, i takes positive integers from 0 to 1,919, and j takes positive integers from 0 to 1,079.

A first pixel corresponding to a light receiving element of the image sensor 13 that detects infrared light having the first wavelength is, for example, a pixel group satisfying i=3N, where N is a positive integer. A second pixel corresponding to a light receiving element that detects infrared light having the second wavelength is, for example, a pixel group satisfying i=3N+1. A third pixel corresponding to a light receiving element that detects infrared light having the third wavelength is a pixel group satisfying i=3N+2.

The control device 6 sets conditions for imaging processing by the light detection unit 3. The control device 6 controls the aperture ratio of a diaphragm provided in the imaging optical system 11. The control device 6 controls timing of start of exposure and timing of end of exposure for the image sensor 13. In this manner, the control device 6 controls the light detection unit 3 to capture the tissue BT irradiated with the detection light L1. The control device 6 acquires captured image data indicating the capture result of the light detection unit 3 from the light detection unit 3. The control device 6 includes a storage unit 14, and stores the captured image data in the storage unit 14. The storage unit 14 stores therein not only the captured image data but also various kinds of information such as data (projection image data) generated by the image generation unit 4 and data indicating settings of the scanning projection apparatus 1.

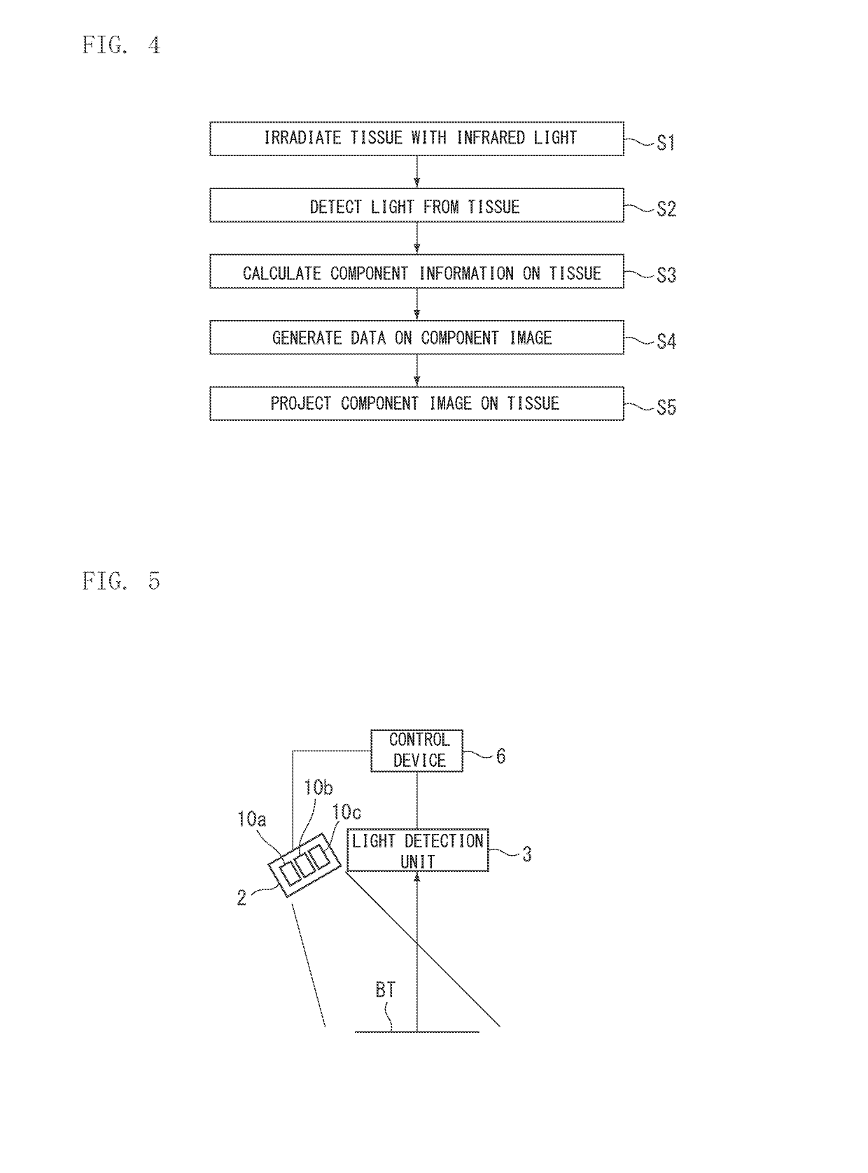

The image generation unit 4 includes a calculation unit 15 and a data generation unit 16. The calculation unit 15 calculates information on components of the tissue BT by using the distribution of light intensity of light (for example, infrared light or fluorescent light) detected by the light detection unit 3 with respect to the wavelength. Now, a method of calculating information on components of the tissue BT will be described. FIG. 3 is a graph showing a distribution D1 of absorbance of a first substance and a distribution D2 of absorbance of a second substance in a near-infrared wavelength region. In FIG. 3, the first substance is lipid and the second substance is water. The vertical axis of the graph in FIG. 3 is the absorbance and the horizontal axis is the wavelength [nm].

A first wavelength λ1 can be set to any desired wavelength. For example, the first wavelength λ1 is set to a wavelength at which the absorbance is relatively small in the distribution of the absorbance of the first substance (lipid) in the near-infrared wavelength region and the absorbance is relatively small in the distribution of the absorbance of the second substance (water) in the near-infrared wavelength region. As one example, in the present embodiment, the first wavelength λ1 is set to about 1150 nm. Infrared light having the first wavelength λ1 is small in energy absorbed by lipid and strong in light intensity radiated from lipid. Infrared light having the first wavelength λ1 is small in energy absorbed by water and strong in light intensity radiated from water.

A second wavelength λ2 can be set to any desired wavelength different from the first wavelength λ1. For example, the second wavelength λ2 is set to a wavelength at which the absorbance of the first substance (lipid) is higher than the absorbance of the second substance (water). As one example, in the present embodiment, the second wavelength λ2 is set to about 1720 nm. When applied to an object (for example, a tissue), infrared light having the second wavelength λ2 is larger in energy absorbed by the object and weaker in light intensity radiated from the object as the proportion of lipid to water included in the object becomes larger. For example, when the proportion of lipid included in a first part of the tissue is larger than that of water, infrared light having the second wavelength λ2 is large in energy absorbed by the first part of the tissue and weak in light intensity radiated from the first part. For example, when the proportion of lipid included in a second part of the tissue is smaller than that of water, infrared light having the second wavelength λ2 is small in energy absorbed by the second part of the tissue and stronger in light intensity radiated from the second part than from the first part.

A third wavelength λ3 can be set to any desired wavelength different from both of the first wavelength λ1 and the second wavelength λ2. For example, the third wavelength λ3 is set to a wavelength at which the absorbance of the second substance (water) is higher than the absorbance of the first substance (lipid). As one example, in the present embodiment, the third wavelength λ3 is set to about 1950 nm. When applied to an object, infrared light having the third wavelength λ3 is larger in energy absorbed by the object and weaker in light intensity radiated from the object as the proportion of water to lipid included in the object becomes larger. Conversely to the case of the above-described second wavelength λ2, for example, when the proportion of lipid included in a first part of the tissue is larger than that of water, infrared light having the third wavelength λ3 is small in energy absorbed by the first part of the tissue and strong in light intensity radiated from the first part. For example, when the proportion of lipid included in a second part of the tissue is smaller than that of water, infrared light having the third wavelength λ3 is large in energy absorbed by the second part of the tissue and is weaker in light intensity radiated from the second part than from the first part.

Referring back to the description with reference to FIG. 1, the calculation unit 15 calculates information on components of the tissue BT by using the captured image data output from the light detection unit 3. In the present embodiment, the wavelength of infrared light detected by each light receiving element of the image sensor 13 is determined by a positional relation between the light receiving element and the infrared filter 12 (first to third filters). The calculation unit 15 calculates the distribution of lipid and the distribution of water included in the tissue BT by using a pixel value P1 corresponding to an output of a light receiving element that detects infrared light having the first wavelength, a pixel value P2 corresponding to an output of a light receiving element that detects infrared light having the second wavelength, and a pixel value P3 corresponding to an output of a light receiving element that detects infrared light having the third wavelength among capture pixels (see FIG. 2).

The pixel P(i,j) in FIG. 2 is a pixel corresponding to a light receiving element that detects infrared light having the first wavelength in the image sensor 13. The pixel P(i+1,j) is a pixel corresponding to a light receiving element that detects infrared light having the second wavelength. The pixel P(i+2,j) is a pixel corresponding to a light receiving element that detects infrared light having the third wavelength in the image sensor 13.

In the present embodiment, the pixel value of the pixel P(i,j) corresponds to the result of detecting infrared light having a wavelength of 1150 nm by the image sensor 13, and the pixel value of the pixel P(i,j) is represented by A1150. The pixel value of the pixel P(i+1,j) corresponds to the result of detecting infrared light having a wavelength of 1720 nm by the image sensor 13, and the pixel value of the pixel P(i+1,j) is represented by A1720. The pixel value of the pixel P(i+2,j) corresponds to the result of detecting infrared light having a wavelength of 1950 nm by the image sensor 13, and the pixel value of the pixel P(i+2,j) is represented by A1950. The calculation unit 15 uses these pixel values to calculate an index Q(i,j) expressed by Expression (1).

Q(i,j)=(A1950−A1150)/(A1720−A1150) (1)

For example, the index Q calculated from Expression (1) is an index indicating the ratio between the amount of lipid and the amount of water in a part of the tissue BT captured by the pixel P(i,j), the pixel P(i+1,j), and the pixel P(i+2,j). As shown in FIG. 3, the absorbance of lipid with respect to infrared light having a wavelength of 1720 nm is larger than the absorbance of water, and hence in a site where the amount of lipid is larger than the amount of water, the value of A1720 becomes smaller, and the value of (A1720−A1150) in Expression (1) becomes smaller. The absorbance of lipid with respect to infrared light having a wavelength of 1950 nm is smaller than the absorbance of water, and hence in a site where the amount of lipid is larger than the amount of water, the value of (A1950−A1150) in Expression (1) becomes larger. In other words, in a site where the amount of lipid is larger than the amount of water, the value of (A1720−A1150) becomes smaller and the value of (A1950−A1150) becomes larger, and hence the index Q(i,j) becomes larger. In this manner, a larger index Q(i,j) indicates a larger amount of lipid, and a smaller index Q(i,j) indicates a larger amount of water.

The calculation unit 15 calculates the index Q(i,j) at the pixel P(i,j) as described above. While changing the values of i and j, the calculation unit 15 calculates indices at other pixels to calculate the distribution of indices. For example, similarly to the pixel P(i,j), a pixel P(i+3,j) corresponds to a light receiving element that detects infrared light having the first wavelength in the image sensor 13, and hence the calculation unit 15 uses the pixel value of the pixel P(i+3,j) instead of the pixel value of the pixel P(i,j) to calculate an index at another pixel. For example, the calculation unit 15 calculates an index Q(i+1,j) by using the pixel value of the pixel P(i+3,j) corresponding to the detection result of infrared light having the first wavelength, the pixel value of a pixel P(i+4,j) corresponding to the detection result of infrared light having the second wavelength, and the pixel value of a pixel P(i+5,j) corresponding to the detection result of infrared light having the third wavelength.

In this manner, the calculation unit 15 calculates an index Q(i,j) of each of a plurality of pixels, thereby calculating the distribution of indices. The calculation unit 15 may calculate an index Q(i,j) for every pixel in the range where pixel values necessary for calculating the indices Q(i,j) are included in captured pixel data. The calculation unit 15 may calculate the distribution of indices Q(i,j) by calculating indices Q(i,j) for part of the pixels and performing interpolation operation by using the calculated indices Q(i,j).

In general, the index Q(i,j) calculated by the calculation unit 15 is not a positive integer. Thus, the data generation unit 16 in FIG. 1 rounds numerical values as appropriate to convert the index Q(i,j) into data in a predetermined image format. For example, the data generation unit 16 generates data on an image about components of the tissue BT by using the result calculated by the calculation unit 15. In the following description, the image about components of the tissue BT is referred to as component image (or projection image) as appropriate. Data on the component image is referred to as component image data (or projection image data).

For the sake of description, the component image is a component image in an HD format as shown in FIG. 2, but there are no limitations on the number of pixels, pixel arrangement (aspect ratio), gray scale of pixel values, and the like of the component image. The component image may be in the same image format as that of the captured image or in an image format different from that of the captured image. The data generation unit 16 performs interpolation processing as appropriate in order to generate data on a component image in an image format different from that of the captured image.

The data generation unit 16 calculates, as the pixel value of the pixel (i,j) in the component image, the value obtained by converting the index Q(i,j) into digital data of 8 bits (256 gray scales). For example, the data generation unit 16 divides the index Q(i,j) by a conversion constant, which is an index corresponding to one gray scale of the pixel value, and rounds the divided value off to the whole number, thereby converting the index Q(i,j) into the pixel value of the pixel (i,j). In this case, the pixel value is calculated so as to satisfy a substantially linear relation to the index.

If the pixel values of three pixels in the captured image are used to calculate an index for one pixel as described above, the number of pixels necessary for calculating an index for a pixel at the edge of the captured image may be insufficient. As a result, the number of indices necessary for calculating the pixel value of a pixel at the edge of a component image is insufficient. In the case where the number of indices necessary for calculating the pixel values of pixels in the component image is insufficient, the data generation unit 16 may calculate the pixel values of pixels in the component image through interpolation. In such a case, the data generation unit 16 may set the pixel value of pixels in the component image that cannot be calculated due to the insufficient number of indices to a predetermined value (for example, 0).

The method of converting the index Q(i,j) into the pixel value can be changed as appropriate. For example, the data generation unit 16 may calculate component image data such that the pixel value and the index have a non-linear relation. The data generation unit 16 may set the value obtained by converting the index calculated with use of the pixel value of the pixel (i,j), the pixel value of the pixel (i+1,j), and the pixel value of the pixel (i+2,j) in the captured image into the pixel value to the pixel value of the pixel (i+1,j).

When the value of the index Q(i,j) is less than the lower limit value of a predetermined range, the data generation unit 16 may determine the pixel value for the index Q(i,j) to be a constant value. The constant value may be the minimum gray scale (for example, 0) of the pixel value. When the value of the index Q(i,j) exceeds the upper limit value of the predetermined range, the data generation unit 16 may determine the pixel value for the index Q(i,j) to be a constant value. The constant value may be the maximum gray scale (for example, 255) of the pixel value or the minimum gray scale (for example, 0) of the pixel value.

The index Q(i,j) calculated by Expression (1) becomes larger as the site has a larger amount of lipid, and hence the pixel value of the pixel (i,j) becomes larger as the site has a larger amount of lipid. For example, a large pixel value generally corresponds to the result that the pixel is displayed brightly, and hence, as the site has a larger amount of lipid, the site is displayed in a more brightly emphasized manner. An operator may request that the site where the amount of water is large be displayed brightly.

Accordingly, in the present embodiment, the scanning projection apparatus 1 has a first mode of displaying information on the amount of the first substance in a brightly emphasized manner and a second mode of displaying information on the amount of the second substance in a brightly emphasized manner. Setting information indicating whether the scanning projection apparatus 1 is set to the first mode or the second mode is stored in the storage unit 14.

When the mode is set to the first mode, the data generation unit 16 generates first component image data obtained by converting the index Q(i,j) into the pixel value of the pixel (i,j). The data generation unit 16 further generates second component image data obtained by converting the reciprocal of the index Q(i,j) into the pixel value of the pixel (i,j). As the amount of water becomes larger in the tissue, the value of the index Q(i,j) becomes smaller and the value of the reciprocal of the index Q(i,j) becomes larger. Thus, the second component image data has high pixel values (gray scales) of pixels corresponding to a site where the amount of water is large.

When the mode is set to the second mode, the data generation unit 16 may calculate a difference value obtained by subtracting the pixel value converted from the index Q(i,j) from a predetermined gray scale as the pixel value of the pixel (i,j). For example, when the pixel value converted from the index Q(i,j) is 50, the data generation unit 16 may calculate 205, which is obtained by subtracting 50 from the maximum gray scale (for example, 255) of the pixel value, as the pixel value of the pixel (i,j).

The image generation unit 4 in FIG. 1 stores the generated component image data in the storage unit 14. The control device 6 supplies the component image data generated by the image generation unit 4 to the projection unit 5, and controls the projection unit 5 to project a component image on the tissue BT in order to emphasize a particular part (for example, the above-described first part or second part) of the tissue BT. The control device 6 controls timing at which the projection unit 5 projects the component image. The control device 6 controls the brightness of the component image projected by the projection unit 5. The control device 6 can control the projection unit 5 to stop projecting the image. The control device 6 can control the start and stop of projection of the component image such that the component image is blinked on the tissue BT for display in order to emphasize a particular part of the tissue BT.

The projection unit 5 is of a scanning projection type that scans the tissue BT with light, and includes a light source 20, the projection optical system 7, and a projection unit controller 21. The light source 20 outputs visible light having a predetermined wavelength different from that of the detection light. The light source 20 includes a laser diode, and outputs laser light as the visible light. The light source 20 outputs laser light having light intensity corresponding to a current supplied from the outside.

The projection optical system 7 guides the laser light output from the light source 20 onto the tissue BT, and scans the tissue BT with the laser light. The projection optical system 7 includes a scanning unit 22 and a wavelength selection mirror 23. The scanning unit 22 can deflect the laser light output from the light source 20 to two directions. For example, the scanning unit 22 is a reflective optical system. The scanning unit 22 includes a first scanning mirror 24, a first drive unit 25 that drives the first scanning mirror 24, a second scanning mirror 26, and a second drive unit 27 that drives the second scanning mirror 26. For example, each of the first scanning mirror 24 and the second scanning mirror 26 is a galvano mirror, a MEMS mirror, or a polygon mirror.

The first scanning mirror 24 and the first drive unit 25 are a horizontal scanning unit that deflects the laser light output from the light source 20 to a horizontal scanning direction. The first scanning mirror 24 is disposed at a position at which the laser light output from the light source 20 enters. The first drive unit 25 is controlled by the projection unit controller 21, and rotates the first scanning mirror 24 on the basis of a drive signal received from the projection unit controller 21. The laser light output from the light source 20 is reflected by the first scanning mirror 24, and is deflected to a direction corresponding to the angular position of the first scanning mirror 24. The first scanning mirror 24 is disposed in an optical path of the laser light output from the light source 20.

The second scanning mirror 26 and the second drive unit 27 are a vertical scanning unit that deflects the laser light output from the light source 20 to a vertical scanning direction. The second scanning mirror 26 is disposed at a position at which the laser light reflected by the first scanning mirror 24 enters. The second drive unit 27 is controlled by the projection unit controller 21, and rotates the second scanning mirror 26 on the basis of a drive signal received from the projection unit controller 21. The laser light reflected by the first scanning mirror 24 is reflected by the second scanning mirror 26, and is deflected to a direction corresponding to the angular position of the second scanning mirror 26. The second scanning mirror 26 is disposed in the optical path of the laser light output from the light source 20.

Each of the horizontal scanning unit and the vertical scanning unit is, for example, a galvano scanner. The vertical scanning unit may have the same configuration as the horizontal scanning unit or may have a different configuration. In general, scanning in the horizontal direction is performed at a higher frequency than scanning in the vertical direction in many cases. Thus, a galvano mirror may be used for scanning in the vertical scanning direction, and a MEMS mirror or a polygon mirror, which operates at a higher frequency than the galvano mirror does, may be used for scanning in the horizontal scanning direction.

The wavelength selection mirror 23 is an optical member that guides the laser light deflected by the scanning unit 22 onto the tissue BT. The laser light reflected by the second scanning mirror 26 is reflected by the wavelength selection mirror 23 to be applied to the tissue BT. In the present embodiment, the wavelength selection mirror 23 is disposed in an optical path between the tissue BT and the light detection unit 3. The wavelength selection mirror 23 is, for example, a dichroic mirror or a dichroic prism. The wavelength selection mirror has characteristics of transmitting detection light output from the light source 10 of the irradiation unit 2 and reflecting visible light output from the light source of the projection unit 5. The wavelength selection mirror 23 has characteristics of transmitting light in an infrared region and reflecting light in a visible region.

Herein, an optical axis 7a of the projection optical system 7 is an axis that is coaxial with (has the same optical axis as) laser light that passes through the center of a scanning area SA scanned with laser light by the projection optical system 7. As one example, the optical axis 7a of the projection optical system 7 is coaxial with laser light that passes through the center of the scanning area SA in the direction of horizontal scanning by the first scanning mirror 24 and the first drive unit 25 and passes through the center of the scanning area SA in the direction of vertical scanning by the second scanning mirror 26 and the second drive unit 27. The optical axis 7a of the projection optical system 7 on the light output side is coaxial with laser light that passes through the center of the scanning area SA in an optical path between the optical member disposed closest to a laser light irradiation target in the projection optical system 7 and the laser light irradiation target. In the present embodiment, the optical axis 7a of the projection optical system 7 on the light output side is coaxial with laser light that passes through the center of the scanning area SA in an optical path between the wavelength selection mirror 23 and the tissue BT.

In the present embodiment, the optical axis 11a of the imaging optical system 11 is coaxial with a rotation center axis of a lens included in the imaging optical system 11. The optical axis 11a of the imaging optical system 11 and the optical axis 7a of the projection optical system 7 on the light output side are set to be coaxial with each other. Thus, even when a capture position for the tissue BT is changed by a user, the scanning projection apparatus 1 in the present embodiment can be used to project the above-described component image on the tissue BT without being displaced. In the present embodiment, the light detection unit 3 and the projection unit 5 are each housed in a casing 30. The light detection unit 3 and the projection unit 5 are each fixed to the casing 30. Thus, positional displacement of the light detection unit 3 and the projection unit 5 is suppressed, and a positional deviation of the optical axis 11a of the imaging optical system 11 and the optical axis of the projection optical system 7 is suppressed.

The projection unit controller 21 controls a current supplied to the light source 20 in accordance with the pixel value. For example, for displaying the pixel (i,j) in the component image, the projection unit controller 21 supplies a current corresponding to the pixel value of the pixel (i,j) to the light source 20. As one example, the projection unit controller 21 modulates the amplitude of the current supplied to the light source 20 in accordance with the pixel value. The projection unit controller 21 controls the first drive unit 25 to control a position at which laser light enters at each time point in the horizontal scanning direction of the laser light scanning area by the scanning unit 22. The projection unit controller 21 controls the second drive unit 27 to control a position at which laser light enters at each time point in the vertical scanning direction of the laser light scanning area by the scanning unit 22. As one example, the projection unit controller 21 controls the light intensity of the laser light output from the light source 20 in accordance with the pixel value of the pixel (i,j), and controls the first drive unit 25 and the second drive unit such that the laser light enters the position corresponding to the pixel (i,j) on the scanning area.

In the present embodiment, the control device 6 is provided with a display device 31 and an input device 32. The display device 31 is, for example, a flat panel display such as a liquid crystal display. The control device 6 can display captured images and setting of operation of the scanning projection apparatus 1, for example, on the display device 31. The control device 6 can display a captured image captured by the light detection unit 3 or an image obtained by subjecting the captured image to image processing on the display device 31. The control device 6 can display a component image generated by the image generation unit 4 or an image obtained by subjecting the component image to image processing on the display device 31. The control device 6 can display a combined image obtained by subjecting a component image and a captured image to combining processing on the display device 31.

For displaying at least one of the captured image or the component image on the display device 31, the display timing may be the same as or different from the timing at which the projection unit 5 projects the component image. For example, the control device 6 may store component image data in the storage unit 14, and supply the component image data stored in the storage unit 14 to the display device 31 when the input device 32 receives an input signal for displaying an image on the display device 31.

The control device 6 may display an image obtained by capturing the tissue BT with an imaging apparatus having sensitivity to the wavelength band of visible light on the display device 31, or may display such an image together with at least one of a component image or a captured image on the display device 31.

Examples of the input device 32 include a switch, a mouse, a keyboard, and a touch panel. The input device 32 can input setting information that sets the operation of the scanning projection apparatus 1. The control device 6 can detect that the input device 32 has been operated. The control device 6 can change the setting of the scanning projection apparatus 1 and controls each unit in the scanning projection apparatus 1 to execute processing in accordance with the information input via the input device 32.

For example, when a user operates the input device 32 to input a designation of the first mode of displaying information on the amount of lipid brightly by the projection unit 5, the control device 6 controls the data generation unit 16 to generate component image data corresponding to the first mode. When the user operates the input device 32 to input a designation of the second mode of displaying information on the amount of water brightly by the projection unit 5, the control device 6 controls the data generation unit 16 to generate component image data corresponding to the second mode. In this manner, the scanning projection apparatus 1 can switch between the first mode and the second mode as the mode of displaying a component image projected by the projection unit 5 in an emphasized manner.

The control device 6 can control the projection unit controller 21 in accordance with an input signal via the input device 32 to start, stop, or restart displaying the component image by the projection unit 5. The control device 6 can control the projection unit controller 21 in accordance with an input signal via the input device 32 to adjust at least one of the color or the brightness of the component image displayed by the projection unit 5. For example, the tissue BT may have a strongly reddish hue due to bloods. In this case, when a component image is displayed in a complementary color (for example, green) of the tissue BT, the tissue BT and the component image can be easily visually distinguished.

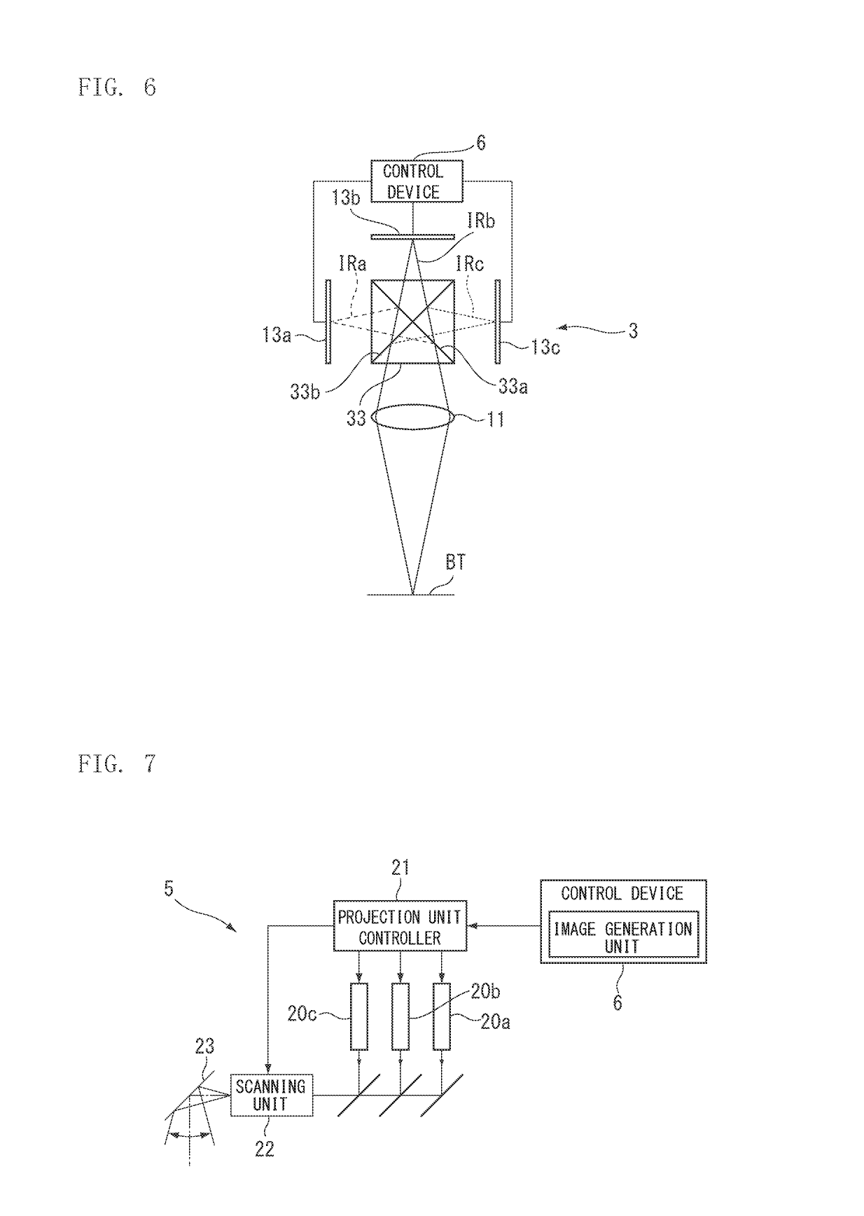

Next, a scanning projection method according to the present embodiment will be described on the basis of the above-described scanning projection apparatus 1. FIG. 4 is a flowchart showing the projection method according to the present embodiment.

At Step S1, the irradiation unit 2 irradiates a biological tissue BT with detection light (for example, infrared light). At Step S2, the light detection unit 3 detects light (for example, infrared light) that is radiated from the tissue BT irradiated with the detection light. At Step S3, the calculation unit 15 in the image generation unit 4 calculates component information on the amount of lipid and the amount of water in the tissue BT. At Step S4, the data generation unit 16 generates data (component image data) on an image (component image) about the tissue BT by using the calculation result of the calculation unit 15. In this manner, at Step S3 and Step S4, the image generation unit 4 generates data on the image about the tissue BT by using the detection result of the light detection unit 3. At Step S5, the projection unit 5 scans the tissue BT with visible light on the basis of the component image data supplied from the control device 6, and projects the component image on the tissue BT through the scanning with the visible light. For example, the scanning projection apparatus 1 in the present embodiment uses two scanning mirrors (for example, the first scanning mirror 24 and the second scanning mirror 26) to sequentially scan the tissue BT with visible light two-dimensionally (in two directions) on the basis of the component image data, thereby projecting the component image on the tissue BT.

The scanning projection apparatus 1 according to the present embodiment uses the scanning unit 22 to scan the tissue BT with laser light, thereby displaying (rendering) an image (for example, a component image) indicating information on the tissue BT directly on the tissue BT. Laser light has high parallelism in general, and the spot size thereof changes less in response to a change in optical path length. Thus, the scanning projection apparatus 1 can project a sharp image with less blur on the tissue BT irrespective of the unevenness of the tissue BT. The scanning projection apparatus 1 can be reduced in size and weight as compared with a configuration in which an image is projected with a projection lens. For example, the scanning projection apparatus 1 can be used as a portable apparatus to improve user operability.

In the scanning projection apparatus 1, the optical axis 11a of the imaging optical system 11 and the optical axis 7a of the projection optical system 7 are set to be coaxial with each other. Thus, even when the relative positions of the tissue BT and the light detection unit 3 are changed, the scanning projection apparatus 1 can reduce a positional deviation between a part of the tissue BT captured by the light detection unit 3 and a part of the tissue BT on which an image is projected by the projection unit 5. For example, the scanning projection apparatus 1 can reduce the occurrence of parallax between an image projected by the projection unit 5 and the tissue BT.

The scanning projection apparatus 1 projects a component image in which a particular site of the tissue BT is emphasized as an image indicating information on the tissue BT. Such a component image can be used, for example, to determine whether the tissue BT has an affected area, such as a tumor. For example, when the tissue BT has a tumor, the ratio of lipid or water included in the tumor area differs from that in a tissue with no tumor. The ratio may differ depending on the type of tumor. Thus, an operator can perform treatment such as incision, excision, and medication to an area with a suspected tumor while viewing the component image on the tissue BT. The scanning projection apparatus 1 can change the color and the brightness of the component image, and hence the component image can be displayed so as to be easily visually distinguished from the tissue BT. In the case where the projection unit 5 irradiates the tissue BT with laser light directly as in the present embodiment, flickering called speckle, which is easily visually recognized, occurs in the component image projected on the tissue BT, and hence a user can easily distinguish the component image from the tissue BT owing to the speckle.

In the scanning projection apparatus 1, the period during which one frame of the component image is projected may be variable. For example, the projection unit 5 can project an image at 60 frames per second, and the image generation unit 4 may generate image data such that an all black image in which all pixels display black may be included between one component image and the next component image. In this case, the component image is blinked and easily visually recognized and is accordingly easily distinguished from the tissue BT.

In the present embodiment, the control device 6 includes an arithmetic circuit such as an ASIC, and executes various kinds of processing such as image computation with the arithmetic circuit. At least a part of the processing executed by the control device 6 may be executed by a computer including a CPU and a memory in accordance with a program. This program is, for example, a program that causes the computer to execute: irradiating a biological tissue BT with detection light; detecting, by the light detection unit 3, light that is radiated from the tissue irradiated with the detection light; generating data on an image about the tissue BT by using a detection result of the light detection unit 3; and scanning the tissue BT with visible light on the basis of the data, and projecting an image on the tissue BT through the scanning with the visible light. This program may be stored in a computer-readable storage medium, such as an optical disc, a CD-ROM, a USB memory, or an SD card, and then provided.

While in the present embodiment, the scanning projection apparatus 1 generates a component image of the tissue BT by using the distribution of light intensity of infrared light radiated from the tissue BT with respect to the wavelength, the component image may be generated by another method. For example, the scanning projection apparatus 1 may generate a component image of the tissue BT by detecting visible light radiated from the tissue BT with the light detection unit 3 and using the detection result of the light detection unit 3.

For example, the scanning projection apparatus 1 shown in FIG. 1 may detect a fluorescent image of the tissue BT added with a fluorescent substance, and generate a component image of the tissue BT on the basis of the detection result. In this case, a fluorescent substance such as indocyanine green (ICG) is added to the tissue BT (affected area) prior to the processing of capturing the tissue BT. For example, the irradiation unit 2 includes a light source that outputs detection light (excitation light) having a wavelength that excites the fluorescent substance added to the tissue BT, and irradiates the tissue BT with the detection light output from the light source. The wavelength of the excitation light is set in accordance with the type of fluorescent substance, and may include the wavelength of infrared light, the wavelength of visible light, or the wavelength of ultraviolet light.

The light detection unit 3 includes a light detector having sensitivity to fluorescent light radiated from the fluorescent substance, and captures an image (fluorescent image) of the tissue BT irradiated with the detection light. For extracting fluorescent light from light radiated from the tissue BT, for example, an optical member having characteristics of transmitting fluorescent light and reflecting at least a part of the light other than the fluorescent light may be used as the wavelength selection mirror 23. A filter having such characteristics may be disposed in an optical path between the wavelength selection mirror 23 and the light detector. The filter may be insertable in and removable from the optical path between the wavelength selection mirror 23 and the light detector, or may be exchangeable in accordance with the type of fluorescent substance, that is, the wavelength of excitation light.

For extracting fluorescent light from light radiated from the tissue BT, a difference between a first captured image in which the tissue BT not irradiated with excitation light is captured and a second captured image in which the tissue BT irradiated with excitation light is captured. For example, the control device 6 in FIG. 1 stops the output of excitation light from the irradiation unit 2, and controls the light detection unit 3 to capture the tissue BT and acquires data on the first captured image from the light detection unit 3. The control device 6 controls the irradiation unit 2 to output excitation light, and controls the light detection unit 3 to capture the tissue BT and acquires data on the second captured image. The image generation unit 4 can extract a fluorescent image by determining the difference between the data on the first captured image and the data on the second captured image.

The image generation unit 4 generates, as component image data, data on an image indicating the extracted fluorescent image. The projection unit 5 projects a component image on the tissue BT on the basis of such component image data. In this manner, the component image indicating the amount and distribution of substances related to the fluorescent substance among components of the tissue BT is displayed on the tissue BT. As described above, the scanning projection apparatus 1 can also generate a component image about substances other than lipid and water.

The scanning projection apparatus 1 may be configured to switch between a mode of generating a component image on the basis of the distribution of light intensity of infrared light radiated from the tissue BT with respect to the wavelength and a mode of generating a component image on the basis of a fluorescent image of the tissue BT. The scanning projection apparatus 1 may project a component image based on the distribution of light intensity of infrared light radiated from the tissue BT with respect to the wavelength and a component image based on the fluorescent image of the tissue BT.

The scanning projection apparatus 1 does not have to generate a component image. For example, the scanning projection apparatus 1 may acquire a component image generated in advance, align the tissue BT and the component image with each other by using a captured image obtained by capturing the tissue BT with the light detection unit 3, and project the component image on the tissue BT.

The scanning projection apparatus 1 does not have to project a component image. For example, an image about the tissue BT is not necessarily an image about components of the tissue BT, but may be an image about a partial position on the tissue BT. For example, the scanning projection apparatus 1 may generate an image indicating the range (position) of a preset site in the tissue BT by using a captured image obtained by capturing the tissue BT with the light detection unit 3, and project the image on the tissue BT. The preset site is, for example, a site in the tissue BT to be subjected to treatment, such as operation and examination. Information on the site may be stored in the storage unit 14 through the operation of the input device 32. The scanning projection apparatus 1 may project an image in a region in the tissue BT different from the region to be detected by the light detection unit 3. For example, the scanning projection apparatus 1 may project an image near the site to be treated in the tissue BT so as not to hinder the viewing of the site.

While in the present embodiment, the irradiation unit 2 outputs infrared light in the wavelength band including the first wavelength, the second wavelength, and the third wavelength, the irradiation unit 2 is not limited to this configuration. A modification of the irradiation unit 2 will be described below.

FIG. 5 is a diagram showing a modification of the irradiation unit 2. The irradiation unit 2 in FIG. 5 includes a plurality of light sources including a light source 10a, a light source 10b, and a light source 10c. The light source 10a, the light source 10b, and the light source 10c each include an LED that outputs infrared light, and output infrared light having different wavelengths. The light source 10a outputs infrared light in a wavelength band that includes the first wavelength but does not include the second wavelength and the third wavelength. The light source 10b outputs infrared light in a wavelength band that includes the second wavelength but does not include the first wavelength and the third wavelength. The light source 10c outputs infrared light in a wavelength band that includes the third wavelength but does not include the first wavelength and the second wavelength.

The control device 6 is capable of controlling turning-on and turning-off of each of the light source 10a, the light source 10b, and the light source 10c. For example, the control device 6 sets the irradiation unit 2 to a first state in which the light source 10a is turned on and the light source 10b and the light source 10c are turned off. In the first state, the tissue BT is irradiated with infrared light having the first wavelength output from the irradiation unit 2. While setting the irradiation unit 2 to the first state, the control device 6 controls the light detection unit 3 to capture the tissue BT and acquires data (captured image data) on an image in which the tissue BT irradiated with infrared light having the first wavelength is captured, from the light detection unit 3.

The control device 6 sets the irradiation unit 2 to a second state in which the light source 10b is turned on and the light source 10a and the light source 10c are turned off. While setting the irradiation unit 2 to the second state, the control device 6 controls the light detection unit 3 to capture the tissue BT, and acquires captured image data on the tissue BT irradiated with infrared light having the second wavelength from the light detection unit 3. The control device 6 sets the irradiation unit 2 to a third state in which the light source 10c is turned on and the light source 10a and the light source 10b are turned off. While setting the irradiation unit 2 to the third state, the control device 6 controls the light detection unit 3 to capture the tissue BT, and acquires captured image data on the tissue BT irradiated with infrared light having the third wavelength from the light detection unit 3.

The scanning projection apparatus 1 can project, on the tissue BT, an image (for example, a component image) indicating information on the tissue BT even with the configuration to which the irradiation unit 2 shown in FIG. 5 is applied. Such a scanning projection apparatus 1 captures the tissue BT with the image sensor 13 (see FIG. 1) for each wavelength band, which makes it easy to secure the resolution.

While in the present embodiment, the light detection unit 3 detects infrared light having the first wavelength, infrared light having the second wavelength, and infrared light having the third wavelength collectively with the same image sensor 13, the light detection unit 3 is not limited to this configuration. A modification of the light detection unit 3 will be described below.

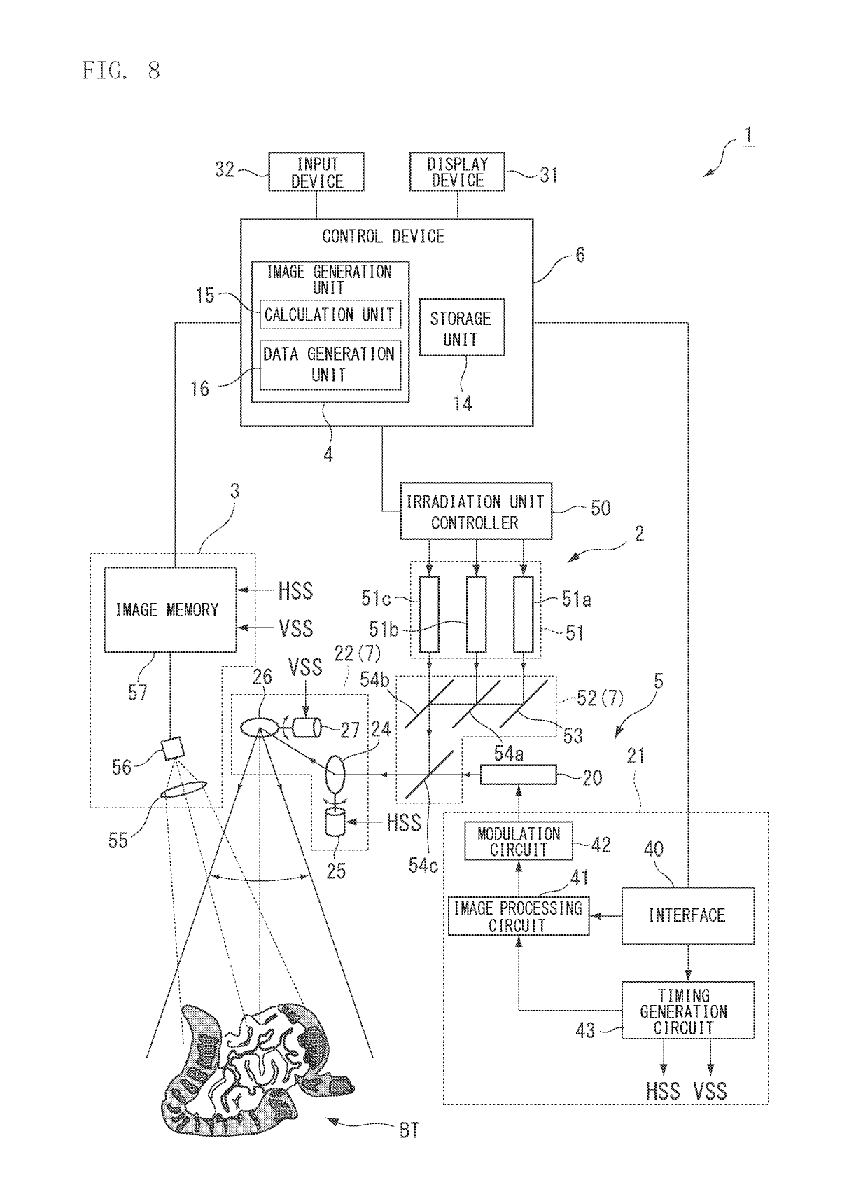

FIG. 6 is a diagram showing a modification of the light detection unit 3. The light detection unit 3 in FIG. 6 includes an imaging optical system 11, a wavelength separation unit 33, and a plurality of image sensors including an image sensor 13a, an image sensor 13b, and an image sensor 13c.

The wavelength separation unit 33 disperses light radiated from the tissue BT depending on the difference in wavelength. The wavelength separation unit 33 in FIG. 6 is, for example, a dichroic prism. The wavelength separation unit 33 includes a first wavelength separation film 33a and a second wavelength separation film 33b. The first wavelength separation film 33a has characteristics of reflecting infrared light IRa having the first wavelength and transmitting infrared light IRb having the second wavelength and infrared light IRc having the third wavelength. The second wavelength separation film 33b is provided so as to intersect with the first wavelength separation film 33a. The second wavelength separation film 33b has characteristics of reflecting the infrared light IRc having the third wavelength and transmitting the infrared light IRa having the first wavelength and the infrared light IRb having the second wavelength.

The infrared light IRa having the first wavelength among the infrared light IR radiated from the tissue BT is reflected and deflected by the first wavelength separation film 33a, and enters the image sensor 13a. The image sensor 13a detects the infrared light IRa having the first wavelength, thereby capturing an image of the tissue BT in the first wavelength. The image sensor 13a supplies data on the captured image (captured image data) to the control device 6.

The infrared light IRb having the second wavelength among the infrared light IR radiated from the tissue BT is transmitted through the first wavelength separation film 33a and the second wavelength separation film 33b, and enters the image sensor 13b. The image sensor 13b detects the infrared light IRb having the second wavelength, thereby capturing an image of the tissue BT in the second wavelength. The image sensor 13b supplies data on the captured image (captured image data) to the control device 6.

The infrared light IRc having the third wavelength among the infrared light IR radiated from the tissue BT is reflected by the second wavelength separation film 33b and deflected to the side opposite to the infrared light IRa having the first wavelength, and enters the image sensor 13c. The image sensor 13c detects the infrared light IRc having the third wavelength, thereby capturing an image of the tissue BT in the third wavelength. The image sensor 13a supplies data on the captured image (captured image data) to the control device 6.

The image sensor 13a, the image sensor 13b, and the image sensor 13c are disposed at positions that are optically conjugate with one another. The image sensor 13a, the image sensor 13b, and the image sensor 13c are disposed so as to have substantially the same optical distance from the imaging optical system 11.

The scanning projection apparatus 1 can project, on the tissue BT, an image indicating information on the tissue BT even with the configuration to which the light detection unit 3 shown in FIG. 6 is applied. Such a light detection unit 3 detects infrared light separated by the wavelength separation unit 33 independently with the image sensor 13a, the image sensor 13b, and the image sensor 13c, which makes it easy to secure the resolution.

The light detection unit 3 may be configured to separate infrared light depending on the difference in wavelength by using, instead of a dichroic prism, a dichroic mirror having the same characteristics as the first wavelength separation film 33a and a dichroic mirror having the same characteristics as the second wavelength separation film 33b. In this case, when the optical path length of any one of the infrared light having the first wavelength, the infrared light having the second wavelength, and the infrared light having the third wavelength is different from the optical path lengths of the other infrared light, a relay lens or the like may be provided to match the optical path lengths.

While the projection unit 5 in the present embodiment projects a monochrome image, the projection unit 5 may project an image with a plurality of colors. FIG. 7 is a diagram showing a modification of the projection unit 5. The projection unit 5 in FIG. 7 includes a laser light source 20a, a laser light source 20b, and a laser light source 20c that output laser light having different wavelengths.

The laser light source 20a outputs laser light in a red wavelength band. The red wavelength band includes 700 nm, and is, for example, 610 nm or more and 780 nm or less. The laser light source 20b outputs laser light in a green wavelength band. The green wavelength band includes 546.1 nm, and is, for example, 500 nm or more and 570 nm or less. The laser light source 20c outputs laser light in a blue wavelength band. The blue wavelength band includes 435.8 nm, and is, for example, 430 nm or more and 460 nm or less.

In the present example, the image generation unit 4 can form a color image based on the amount or proportion of components as an image to be projected by the projection unit 5. For example, the image generation unit 4 generates green image data such that the gray-scale value of green becomes higher as the amount of lipid becomes larger. The image generation unit 4 generates blue image data such that the gray-scale value of blue becomes higher as the amount of water becomes larger. The control device 6 supplies component image data including the green image data and the blue image data generated by the image generation unit 4 to the projection unit controller 21.

The projection unit controller 21 drives the laser light source 20b by using green image data among the component image data supplied from the control device 6. For example, the projection unit controller 21 increases the current supplied to the laser light source 20b so that light intensity of green laser light output from the laser light source 20b may be increased as the pixel value defined by green image data becomes higher. Similarly, the projection unit controller 21 drives the laser light source 20c by using blue image data among the component image data supplied from the control device 6.

The scanning projection apparatus 1 to which such a projection unit 5 is applied can display a part where the amount of lipid is large in green in a brightly emphasized manner, and display a part where the amount of water is large in blue in a brightly emphasized manner. The scanning projection apparatus 1 may display a part where both the amount of lipid and the amount of water are large in red brightly, or may display the amount of a third substance different from lipid and water in red.

While in FIG. 1 and others, the light detection unit 3 detects light passing through the wavelength selection mirror 23 and the projection unit 5 projects a component image with light reflected by the wavelength selection mirror 23, the light detection unit 3 is not limited to this configuration. For example, the light detection unit 3 may detect light reflected by the wavelength selection mirror 23 and the projection unit 5 may project a component image with light passing through the wavelength selection mirror 23. The wavelength selection mirror 23 may be a part of the imaging optical system 11, or may be a part of the projection optical system 7. The optical axis of the projection optical system 7 does not have to be coaxial with the optical axis of the imaging optical system 11.

### Second Embodiment

Next, a second embodiment will be described. In the second embodiment, the same configuration as in the above-described embodiment is denoted by the same reference symbol and description thereof is simplified or omitted.

FIG. 8 is a diagram showing a scanning projection apparatus 1 according to the second embodiment. In the second embodiment, a projection unit controller 21 includes an interface 40, an image processing circuit 41, a modulation circuit 42, and a timing generation circuit 43. The interface 40 receives image data from the control device 6. The image data includes gray-scale data indicating pixel values of pixels, and synchronization data that defines the refresh rate and the like. The interface extracts gray-scale data from the image data, and supplies the gray-scale data to the image processing circuit 41. The interface 40 extracts synchronization data from the image data, and supplies the synchronization data to the timing generation circuit 43.

The timing generation circuit 43 generates timing signals representing operation timings of the light source 20 and the scanning unit 22. The timing generation circuit 43 generates a timing signal in accordance with the image resolution, the refresh rate (frame rate), and the scanning method. For the sake of description, the image is in a full HD format, and scanning with light has no time (blanking time) from when the rendering of one horizontal scanning line is finished to when the rendering of the next horizontal scanning line is started.

The full HD image has horizontal scanning lines, in each of which 1,920 pixels are arranged, and 1,080 horizontal scanning lines are arranged in the vertical scanning direction. When an image is displayed at a refresh rate of 30 Hz, the cycle of scanning in the vertical scanning direction is about 33 milliseconds (1/30 second). For example, the second scanning mirror 26 that scans in the vertical scanning direction turns from one end to the other end of the turning range in about 33 milliseconds, thereby scanning an image for one frame in the vertical scanning direction. The timing generation circuit 43 generates a signal that defines the time at which the second scanning mirror 26 starts to render the first horizontal scanning line for each frame as the vertical scanning signal VSS. The vertical scanning signal VSS is, for example, a waveform that rises with a cycle of about 33 milliseconds.

The rendering time (lighting time) per horizontal scanning line is about 31 microseconds (1/30/1080 second). For example, the first scanning mirror 24 turns from one end to the other end of the turning range in about 31 microseconds, thereby performing scanning corresponding to one horizontal scanning line. The timing generation circuit 43 generates a signal that defines the time at which the first scanning mirror 24 starts scanning of each horizontal scanning line as the horizontal scanning signal HSS. The horizontal scanning signal HSS is, for example, a waveform that rises with a cycle of about 31 microseconds.

The lighting time per pixel is about 16 nanoseconds (1/30/1080/1920 second). For example, the light intensity of laser light output from the light source 20 is switched with a cycle of about 16 nanoseconds in accordance with the pixel value, thereby displaying each pixel. The timing generation circuit 43 generates a lighting signal that defines the timing at which the light source 20 is turned on. The lighting signal is, for example, a waveform that rises with a cycle of about 16 nanoseconds.

The timing generation circuit 43 supplies the generated horizontal scanning signal HSS to the first drive unit 25. The first drive unit 25 drives the first scanning mirror 24 in accordance with the horizontal scanning signal HSS. The timing generation circuit 43 supplies the generated vertical scanning signal VSS to the second drive unit 27. The second drive unit 27 drives the second scanning mirror 26 in accordance with the vertical scanning signal VSS.

The timing generation circuit 43 supplies the generated horizontal scanning signal HSS and vertical scanning signal VSS and the lighting signal to the image processing circuit 41. The image processing circuit 41 performs various kinds of image processing, such as gamma processing, on the gray-scale data in the image data. The image processing circuit 41 adjusts the gray-scale data on the basis of the timing signal supplied from the timing generation circuit 43 so that the gray-scale data are sequentially output to the modulation circuit 42 in the order that conforms to the scanning method of the scanning unit 22. For example, the image processing circuit 41 stores the gray-scale data in a frame buffer, reads out the gray-scale data in the order of pixels that display the pixel values included in the gray-scale data, and outputs the gray-scale data to the modulation circuit 42.

The modulation circuit 42 adjusts the output of the light source 20 such that the intensity of laser light radiated from the light source 20 may change with time correspondingly to the gray scale for each pixel. In the present embodiment, the modulation circuit 42 generates a waveform signal whose amplitude changes in accordance with the pixel value, and drives the light source 20 on the basis of the waveform signal. Accordingly, the current supplied to the light source 20 changes with time in accordance with the pixel value, and the light intensity of laser light emitted from the light source 20 changes with time in accordance with the pixel value. In this manner, the timing signal generated by the timing generation circuit 43 is used to synchronize the light source 20 and the scanning unit 22.

In the second embodiment, the irradiation unit 2 includes an irradiation unit controller 50, a light source 51, and a projection optical system 7. The irradiation unit controller 50 controls turning-on and turning-off of the light source 51. The light source 51 outputs laser light as detection light. The irradiation unit 2 deflects the laser light output from the light source 51 to predetermined two directions (for example, first direction and second direction) by the projection optical system 7, and scans the tissue BT with the laser light.

The light source 51 includes a plurality of laser light sources including a laser light source 51a, a laser light source 51b, and a laser light source 51c. The laser light source 51a, the laser light source 51b, and the laser light source 51c each include a laser element that outputs infrared light, and output infrared light having different wavelengths. The laser light source 51a outputs infrared light in a wavelength band that includes a first wavelength but does not include a second wavelength and a third wavelength. The laser light source 51b outputs infrared light in a wavelength band that includes the second wavelength but does not include the first wavelength and the third wavelength. The laser light source 51c outputs infrared light in a wavelength band that includes the third wavelength but does not include the first wavelength and the second wavelength.

The irradiation unit controller 50 supplies a drive current for the laser element of each of the laser light source 51a, the laser light source 51b, and the laser light source 51c. The irradiation unit controller 50 supplies the current to the laser light source 51a to turn on the laser light source 51a, and stops the supply of the current to the laser light source 51a to turn off the laser light source 51a. The irradiation unit controller 50 is controlled by the control device 6 to start or stop the supply of the current to the laser light source 51a. For example, the control device 6 controls timing of turning on or off the laser light source 51a via the irradiation unit controller 50. Similarly, the irradiation unit controller 50 turns on or off each of the laser light source 51b and the laser light source 51c. The control device 6 controls timing of turning on or off each of the laser light source 51b and the laser light source 51c.

The projection optical system 7 includes a light guide unit 52 and a scanning unit 22. The scanning unit 22 has the same configuration as in the first embodiment, and includes the first scanning mirror 24 and first drive unit (horizontal scanning unit) and the second scanning mirror and second drive unit 27 (vertical scanning unit). The light guide unit 52 guides detection light output from each of the laser light source 51a, the laser light source 51b, and the laser light source 51c to the scanning unit 22 so that the detection light may pass through the same optical path of visible light output from the light source 20 of the projection unit 5.

The light guide unit 52 includes a mirror 53, a wavelength selection mirror 54a, a wavelength selection mirror 54b, and a wavelength selection mirror 54c. The mirror 53 is arranged at a position at which the detection light having the first wavelength output from the laser light source 51a enters.

The wavelength selection mirror 54a is arranged at a position at which the detection light having the first wavelength reflected by the mirror 53 and the detection light having the second wavelength output from the laser light source 51b enter. The wavelength selection mirror 54a has characteristics of transmitting detection light having the first wavelength and reflecting detection light having the second wavelength.

The wavelength selection mirror 54b is arranged at a position at which the detection light having the first wavelength transmitted through the wavelength selection mirror 54a, the detection light having the second wavelength reflected by the wavelength selection mirror 54b, and the detection light having the third wavelength output from the laser light source 51c enter. The wavelength selection mirror 54b has characteristics of reflecting detection light having the first wavelength and detection light having the second wavelength and transmitting detection light having the third wavelength.

The wavelength selection mirror 54c is arranged at a position at which the detection light having the first wavelength and the detection light having the second wavelength, which are reflected by the wavelength selection mirror 54b, the detection light having the third wavelength that is transmitted through the wavelength selection mirror 54b, and the visible light output from the light source 20 enter. The wavelength selection mirror 54c has characteristics of reflecting detection light having the first wavelength, detection light having the second wavelength, and detection light having the third wavelength, and transmitting visible light.

The detection light having the first wavelength, the detection light having the second wavelength, and the detection light having the third wavelength, which are reflected by the wavelength selection mirror 54c, and the visible light transmitted through the wavelength selection mirror 54c pass through the same optical path to enter the first scanning mirror 24 in the scanning unit 22. The detection light having the first wavelength, the detection light having the second wavelength, and the detection light having the third wavelength, which enter the scanning unit 22, are each deflected by the scanning unit 22 similarly to the visible light for image projection. In this manner, the irradiation unit 2 can use the scanning unit 22 to scan the tissue BT with each of the detection light having the first wavelength, the detection light having the second wavelength, and the detection light having the third wavelength. Thus, the scanning projection apparatus 1 in the present embodiment has both of the scanning imaging function and the scanning image projection function.

In the present embodiment, the light detection unit 3 detects light that is radiated from the tissue BT scanned with laser by the irradiation unit 2. The light detection unit 3 associates the light intensity of the detected light with positional information on laser light from the irradiation unit 2, thereby detecting the spatial distribution of light intensity of light radiated from the tissue BT in the range where the irradiation unit 2 scans the tissue BT with laser light. The light detection unit 3 includes a condenser lens 55, a light sensor 56, and an image memory 57.

The light sensor 56 includes a photodiode, such as a silicon PIN photodiode or a GaAs photodiode. Electric charges corresponding to the light intensity of incident light are generated in the photodiode of the light sensor 56. The light sensor 56 outputs the electric charges generated in the photodiode as a detection signal in a digital format. For example, the light sensor 56 has one or several pixels, which is smaller than the number of pixels of an image sensor. Such a light sensor 56 is compact and low in cost as compared with a general image sensor.

The condenser lens 55 condenses at least a part of the light radiated from the tissue BT to the photodiode of the light sensor 56. The condenser lens 55 may not form an image of the tissue BT (detection light irradiation region). Specifically, the condenser lens 55 may not make the detection light irradiation region and the photodiode of the light sensor 56 be optically conjugate with each other. Such a condenser lens 55 can be reduced in size and weight and is low in cost as compared with a general imaging lens (image forming optical system).

The image memory 57 stores therein digital signals output from the light sensor 56. The image memory 57 is supplied with a horizontal scanning signal HSS and a vertical scanning signal VSS from the projection unit controller 21. The image memory 57 uses the horizontal scanning signal HSS and the vertical scanning signal VSS to convert the signals output from the light sensor 56 into data in an image format.

For example, the image memory 57 uses a detection signal that is output from the light sensor 56 in a period from the rise to the fall of the vertical scanning signal VSS as image data for one frame. The image memory 57 starts to store therein a detection signal from the light sensor in synchronization with the rise of the vertical scanning signal VSS. The image memory 57 uses a detection signal that is output from the light sensor 56 in a period from the rise to the fall of the horizontal scanning signal HSS as data for one horizontal scanning line. The image memory 57 starts to store therein the data for the horizontal scanning line in synchronization with the rise of the vertical scanning signal VSS. The image memory 57 ends to store therein the data for the horizontal scanning line in synchronization with the fall of the vertical scanning signal VSS. The image memory 57 stores therein the data for each horizontal scanning line repeatedly as often as the number of horizontal scanning lines, thereby storing therein data in an image format corresponding to an image in one frame. Such data in an image format is referred to as “detected image data” as appropriate in the following description. The detected image data corresponds to the captured image data described in the first embodiment. The light detection unit 3 supplies the detected image data to the control device 6.

The control device 6 controls the wavelength of detection light from the irradiation unit 2. The control device 6 controls the irradiation unit controller 50 to control the wavelength of detection light to be output from the light source 51. The control device 6 supplies a control signal that defines the timing of turning on or off the laser light source 51a, the laser light source 51b, and the laser light source 51c to the irradiation unit controller 50. The irradiation unit controller 50 selectively turns on the laser light source 51a, the laser light source 51b, and the laser light source 51c in accordance with the control signal supplied from the control device 6.

For example, the control device 6 turns on the laser light source 51a and turns off the laser light source 51b and the laser light source 51c. In this case, laser light having the first wavelength is output from the light source 51 as detection light, and laser light having the second wavelength and laser light having the third wavelength are not output. In this manner, the control device 6 can switch the wavelength of detection light to be output from the light source 51 among the first wavelength, the second wavelength, and the third wavelength.

The control device 6 controls the light detection unit 3 to detect light that is radiated from the tissue BT in a first period during which the irradiation unit 2 irradiates the tissue BT with light having the first wavelength. The control device 6 controls the light detection unit 3 to detect light that is radiated from the tissue BT in a second period during which the irradiation unit 2 irradiates the tissue BT with light having the second wavelength. The control device 6 controls the light detection unit 3 to detect light that is radiated from the tissue BT in a third period during which the irradiation unit 2 irradiates the tissue BT with light having the third wavelength. The control device 6 controls the light detection unit 3 to output the detection result of the light detection unit 3 in the first period, the detection result of the light detection unit 3 in the second period, and the detection result of the light detection unit 3 in the third period separately to the image generation unit 4.

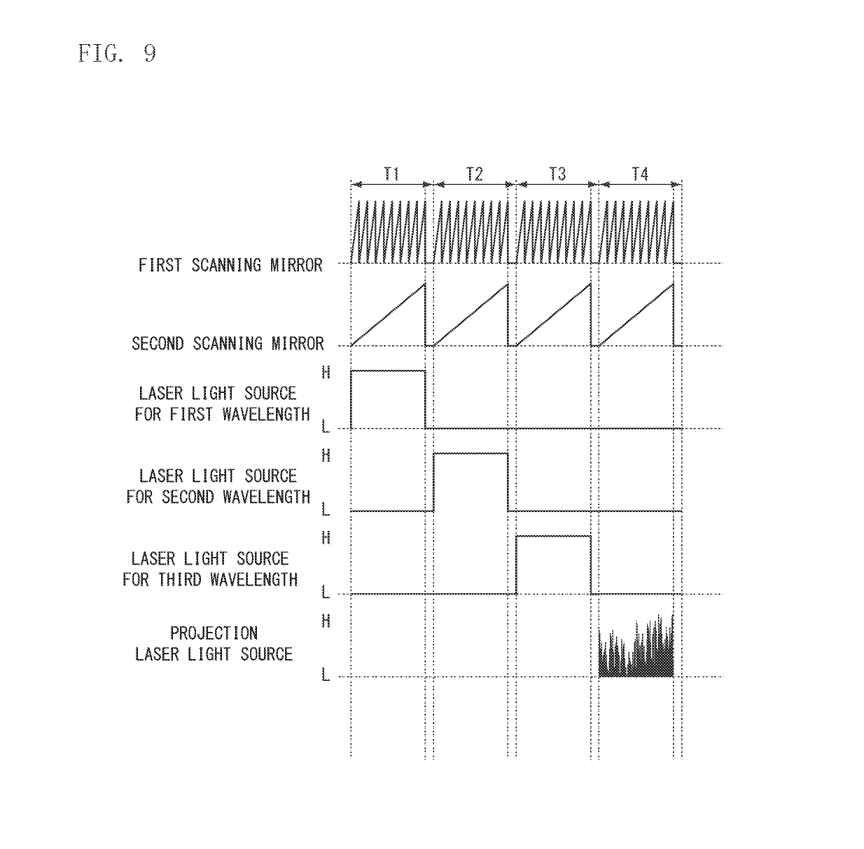

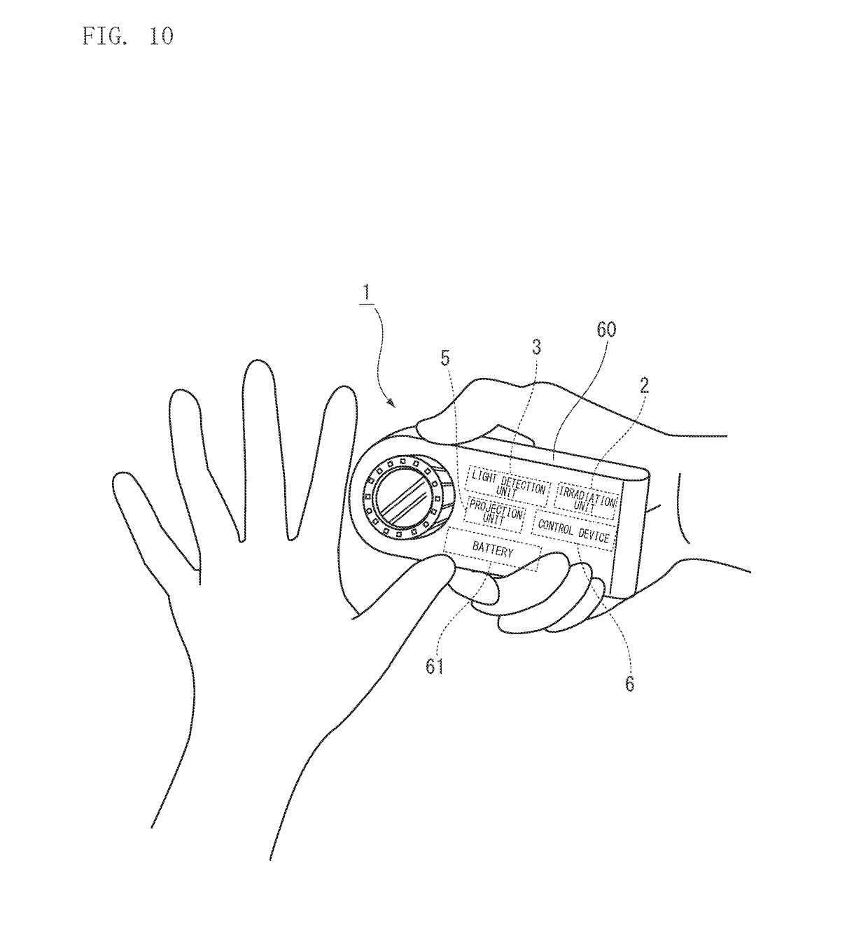

FIG. 9 is a timing chart showing an example of operation of the irradiation unit 2 and the projection unit 5. FIG. 9 shows an angular position of the first scanning mirror 24, an angular position of the second scanning mirror 26, and electric power supplied to each light source. A first period T1 corresponds to a display period for one frame, and the length thereof is about 1/30 second when the refresh rate is 30 Hz. The same is applied to a second period T2, a third period T3, and a fourth period T4.

In the first period T1, the control device 6 turns on the laser light source 51a for the first wavelength. In the first period T1, the control device 6 turns off the laser light source 51b for the second wavelength and the laser light source 51c for the third wavelength.

In the first period T1, the first scanning mirror 24 and the second scanning mirror 26 operate in the same conditions as when the projection unit 5 projects an image. In the first period T1, the first scanning mirror 24 turns from one end to the other end of the turning range repeatedly as often as the number of horizontal scanning lines. For the angular position of the first scanning mirror 24, the unit waveform from one rise to the next rise corresponds to an angular position for scanning of one horizontal scanning line. For example, when an image projected by the projection unit 5 is in the full HD format, the first period T1 includes 1,080 cycles of unit waveforms for the angular position of the first scanning mirror 24. In the first period T1, the second scanning mirror 26 turns once from one end to the other end of the turning range.

Through the operation of the scanning unit 22 as described above, laser light having the first wavelength output from the laser light source 51a scans the entire scanning area on the tissue BT. The control device 6 acquires first detected image data, which corresponds to the result detected by the light detection unit 3 in the first period T1, from the light detection unit 3.

In the second period T2, the control device 6 turns on the laser light source 51b for the second wavelength. In the second period T2, the control device 6 turns off the laser light source 51a for the first wavelength and the laser light source 51c for the third wavelength. In the second period T2, the first scanning mirror 24 and the second scanning mirror 26 operate similarly to the first period T1. In this manner, laser light having the second wavelength output from the laser light source 51b scans the entire scanning area on the tissue BT. The control device 6 acquires second detected image data, which corresponds to the result detected by the light detection unit 3 in the second period T2, from the light detection unit 3.

In the third period T3, the control device 6 turns on the laser light source 51c for the third wavelength. In the third period T3, the control device 6 turns off the laser light source 51a for the first wavelength and the laser light source 51b for the second wavelength. In the third period T3, the first scanning mirror 24 and the second scanning mirror 26 operate similarly to the first period T1. In this manner, laser light having the third wavelength output from the laser light source 51c scans the entire scanning area on the tissue BT. The control device 6 acquires third detected image data, which corresponds to the result detected by the light detection unit 3 in the third period T3, from the light detection unit 3.

The image generation unit 4 shown in FIG. 8 uses the first detected image data, the second detected image data, and the third detected image data to generate a component image, and supplies component image data to the projection unit 5. The image generation unit 4 generates the component image by using the detected image data instead of captured image data described in the first embodiment. For example, the calculation unit 15 calculates information on components of the tissue BT by using a temporal change in light intensity of light detected by the light detection unit 3.

In the fourth period T4, the projection unit controller 21 shown in FIG. 8 uses the component image data supplied from the control device 6 to supply a drive power waveform whose amplitude changes with time in accordance with the pixel value to the projection light source 20 and to control the scanning unit 22. In this manner, the projection unit 5 projects a component image on the tissue BT in the fourth period T4.

The scanning projection apparatus 1 according to the present embodiment detects light radiated from the tissue BT with the light sensor 56 along with laser-scanning of the tissue BT with detection light, and acquires detected image data corresponding to captured image data on the tissue BT. Such a light sensor 56 may have a smaller number of pixels than an image sensor. Thus, the scanning projection apparatus 1 can be reduced in size, weight, and cost. The light receiving area of the light sensor 56 can be easily increased to be larger than the light receiving area of one pixel of an image sensor, and hence the detection accuracy of the light detection unit 3 can be increased.

In the present embodiment, the irradiation unit 2 includes a plurality of light sources that output light having different wavelengths, and irradiates the tissue BT with detection light while temporally switching a light source to be turned on among the light sources. Thus, the amount of light having wavelengths not to be detected by the light detection unit 3 can be reduced as compared with a configuration in which the tissue BT is irradiated with detection light having a broad wavelength. Consequently, for example, energy per unit time to be applied to the tissue BT by detection light can be reduced, and the increase in temperature of the tissue BT by detection light L1 can be suppressed. The light intensity of detection light can be enhanced without increasing the energy per unit time to be applied to the tissue BT by detection light, and hence the detection accuracy of the light detection unit 3 can be increased.

In the example in FIG. 9, the first period T1, the second period T2, and the third period T3 are an irradiation period during which the irradiation unit 2 irradiates the tissue BT with detection light and are a detection period during which the light detection unit 3 detects light radiated from the tissue BT. The projection unit 5 does not project an image in at least a part of the irradiation period and the detection period. Thus, the projection unit 5 can display an image such that the projected image is viewed in a blinking manner. Consequently, a user can easily distinguish a component image and others from the tissue BT.

The projection unit 5 may project an image in at least a part of the irradiation period and the detection period. For example, the scanning projection apparatus 1 may generate a first component image by using the result detected by the light detection unit 3 in a first detection period, and project the first component image on the tissue BT in at least a part of a second detection period after the first detection period. For example, in a period during which the projection unit 5 projects an image, the irradiation unit 2 may irradiate the tissue BT with detection light and the light detection unit 3 may detect light. In a period during which the projection unit 5 displays an image for a first frame, the image generation unit 4 may generate data on an image for a second frame to be projected after the first frame. The image generation unit 4 may generate data on the image for the second frame by using the result detected by the light detection unit 3 in the period during which the image for the first frame is displayed. The projection unit 5 may project the image for the second frame next to the image for the first frame as described above.

While in the present embodiment, the irradiation unit irradiates the tissue BT with detection light by selectively switching a light source to be turned on among a plurality of light sources, the irradiation unit 2 may irradiate the tissue BT with detection light by concurrently turning on two or more light sources among a plurality of light sources. For example, the irradiation unit controller 50 may control the light source 51 such that all of the laser light source 51a, the laser light source 51b, and the laser light source 51c are turned on. In this case, the light detection unit 3 may detect light radiated from the tissue BT for each wavelength through wavelength separation as shown in FIG. 6.

### Third Embodiment

Next, a scanning projection apparatus according to a third embodiment will be described. In the present embodiment, the same configuration as in the above-described embodiments is denoted by the same reference symbol and description thereof is simplified or omitted.

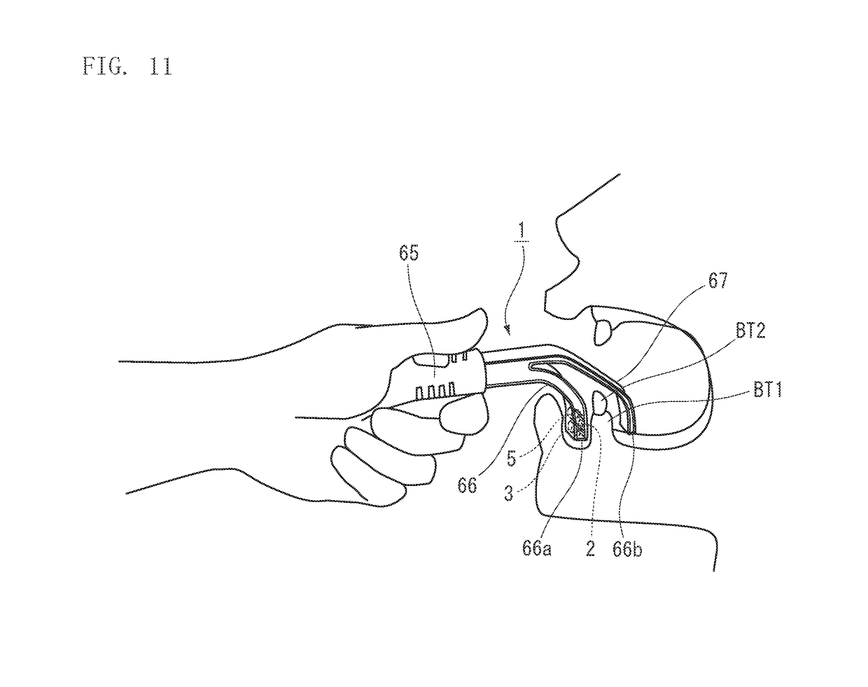

FIG. 10 is a diagram showing a scanning projection apparatus 1 according to the third embodiment. The scanning projection apparatus 1 is a portable apparatus, such as a dermoscope. The scanning projection apparatus 1 has a body 60 having a shape that allows a user to hold in his/her hand. The body 60 is a casing in which the irradiation unit 2, the light detection unit 3, and the projection unit 5 shown in FIG. 8 and others are provided. In the present embodiment, the body 60 is provided with the control device 6 and a battery 61. The battery 61 supplies electric power consumed by each unit in the scanning projection apparatus 1.

In the present embodiment, the scanning projection apparatus 1 may not include the battery 61. For example, the scanning projection apparatus 1 may be supplied with electric power in a wired manner via a power supply cable, or may be supplied with electric power in a wireless manner. The control device 6 may not be provided in the body 60, and may be communicably connected to each unit via a communication cable or in a wireless manner. The irradiation unit 2 may not be provided in the body 60, and may be fixed to a support member such as a tripod. At least one of the light detection unit 3 or the projection unit 5 may be provided to a member different from the body 60.

The scanning projection apparatus 1 in the present embodiment may be a wearable projection apparatus including a mount unit (for example, a belt) that can be directly mounted to the user's body, such as the head or the arm (including fingers). The scanning projection apparatus 1 in the present embodiment may be provided in a medical support robot for operation, pathology, or examination, or may include a mount unit that can be mounted to a hand unit of the medical support robot.

### Fourth Embodiment

Next, a scanning projection apparatus according to a fourth embodiment will be described. In the present embodiment, the same configuration as in the above-described embodiments is denoted by the same reference symbol and description thereof is simplified or omitted.

FIG. 11 is a diagram showing a scanning projection apparatus 1 according to the fourth embodiment. The scanning projection apparatus 1 is used for treatment such as examination and observation of dentition. The scanning projection apparatus 1 includes a base 65, a holding plate 66, a holding plate 67, the irradiation unit 2, the light detection unit 3, and the projection unit 5. The base 65 is a portion to be gripped by a user or a robot hand. The control device 6 shown in FIG. 1 and others is housed inside the base 65, for example, but at least a part of the control device 6 may be provided to a member different from the base 65.

The holding plate 66 and the holding plate 66 are formed by branching a single member extending from one end of the base 65 into two parts from the middle and bending the two parts in the same direction. The interval between a distal end portion 66a of the holding plate 66 and a distal end portion 67a of the holding plate 67 is set to a distance that allows a gum BT1, for example, to be located between the distal end portion 66a and the distal end portion 67a. At least one of the holding plate 66 and the holding plate 67 may be formed of, for example, a deformable material so that the interval between the distal end portion 66a and the distal end portion 67a can be changed. Each of the irradiation unit 2, the light detection unit 3, and the projection unit 5 is provided to the distal end portion 66a of the holding plate 66.

The scanning projection apparatus 1 is configured such that the holding plate 66 and the holding plate 67 are inserted from the mouse of an examinee, and the gum BT1 or a tooth BT2, which is a treatment target, is disposed between the distal end portion 66a and the distal end portion 67a. Subsequently, the irradiation unit 2 at the distal end portion 66a irradiates the gum BT1, for example, with detection light, and the light detection unit 3 detects light radiated from the gum BT1. The projection unit 5 projects an image indicating information on the gum BT1 on the gum BT1. Such a scanning projection apparatus 1 can be utilized for examination and observation of lesions that change the distribution of blood or water, such as edema and inflammation, by projecting a component image indicating the amount of water included in the gum BT1, for example. The portable scanning projection apparatus 1 can be applied to an endoscope as well as the example shown in FIG. 10 or FIG. 11.

### [Surgery Support System]

Next, a surgery support system (medical support system) will be described. In the present embodiment, the same configuration as in the above-described embodiments is denoted by the same reference symbol and description thereof is simplified or omitted.

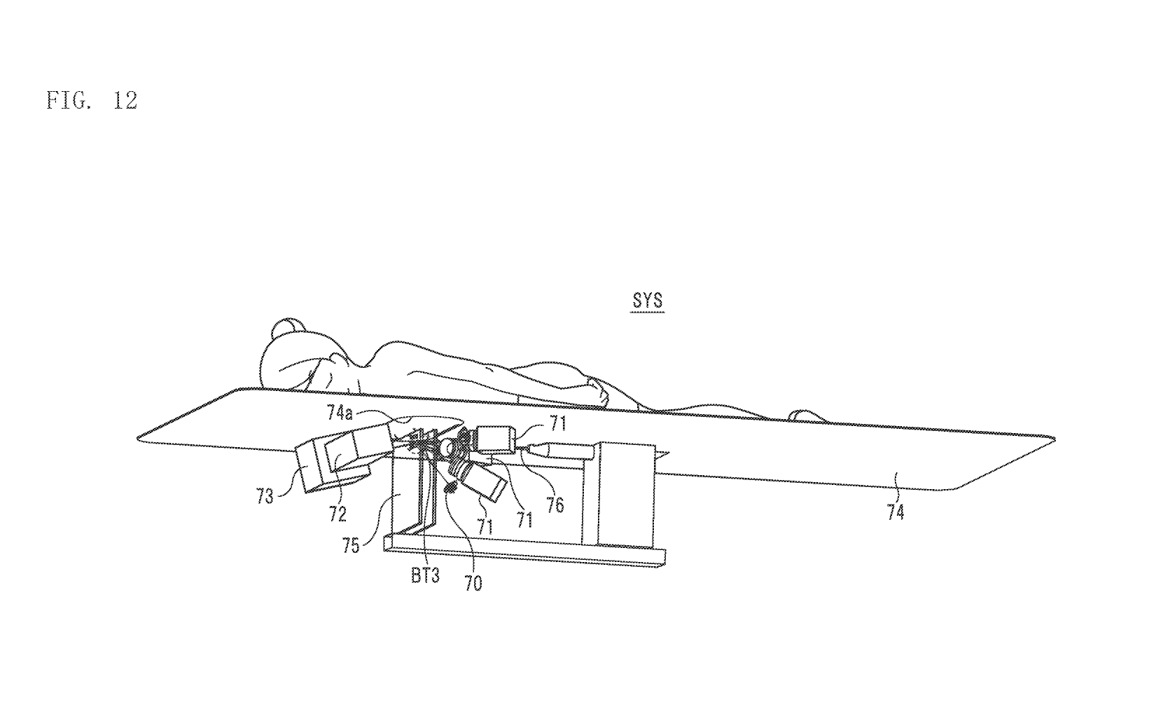

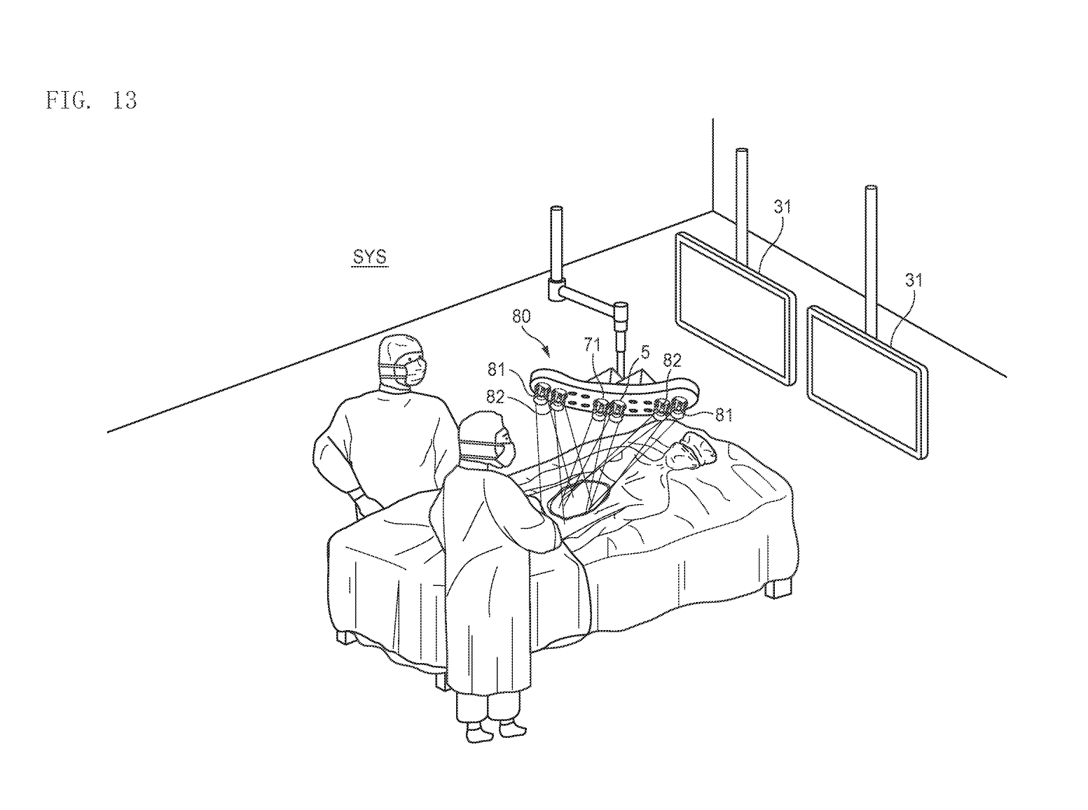

FIG. 12 is a diagram showing an example of a surgery support system SYS according to the present embodiment. The surgery support system SYS is a mammotome using the scanning projection apparatus described in the above-described embodiments. The surgery support system SYS includes, as the scanning projection apparatus, a lighting unit 70, an infrared camera 71, a laser light source 72, and a galvano scanner 73. The lighting unit 70 is an irradiation unit that irradiates a tissue such as a breast tissue with detection light. The infrared camera 71 is a light detection unit that detects light radiated from the tissue. The laser light source 72 and the galvano scanner are a projection unit that projects an image (for example, a component image) indicating information on the tissue. The projection unit projects an image generated by the control device (not shown) by using the detection result of the light detection unit.

The surgery support system SYS also includes a bed 74, a transparent plastic plate 75, and a perforation needle 76. The bed 74 is a bed on which an examinee lies with his or her face down. The bed 74 has an aperture 74a through which a breast BT3 (tissue) of the examinee as the subject is exposed downward. The transparent plastic plate 75 is used to sandwich both sides of the breast BT3 to flatten the breast BT3. The perforation needle 76 is an operation device capable of treating the tissue. The perforation needle 76 is inserted into the breast BT3 in a core needle biopsy to take a sample.

The infrared camera 71, the lighting unit 70, the laser light source 72, and the galvano scanner 73 are disposed below the bed 74. The infrared camera 71 is disposed with the transparent plastic plate 75 located between the infrared camera 71 and the galvano scanner 73. The infrared cameras 71 and the lighting units 70 are disposed so as to form a spherical shape.

As shown in FIG. 12, the breast BT3 is flattened by pressing the transparent plastic plate 75 against both sides thereof, and in this state, the lighting unit 70 outputs infrared light having a predetermined wavelength so that the infrared camera 71 captures an image. In this manner, the infrared camera 71 acquires an image of the breast BT3 with infrared light reflected from the lighting unit 70. The laser light source 72 and the galvano scanner project a component image generated from the image captured by the infrared camera 71.

In a general core needle biopsy, a perforation needle (core needle) is inserted while measuring the depth of the needle using ultrasonic echo. A breast generally includes tissues with a large amount of lipid, but when a breast cancer occurs, the amount of water in the breast cancer area may differ from the amount in other parts.

The surgery support system SYS can insert the perforation needle 76 into the breast BT3 to take a sample while projecting a component image of the breast BT3 by the laser light source 72 and the galvano scanner 73. For example, an operator can insert the perforation needle 76 into a part of the breast BT3 where the amount of water is different from those in other parts while observing a component image projected on the breast BT3.

With the surgery support system SYS according to the present embodiment, the infrared mammotome using the difference between infrared spectrums is used in a core needle biopsy. Thus, a sample can be taken on the basis of the spatial recognition of an accurate tissue image. Imaging using infrared light, which does not cause X-ray exposure, has an advantage that it can be usually used in obstetrics and gynecology, regardless of whether the patient is pregnant.

Next, another example of the surgery support system is described. FIG. 13 is a diagram showing another example of the surgery support system SYS. The surgery support system SYS is used for a laparotomy or other operations. The surgery support system SYS includes an operation device (not shown) capable of treating a tissue to be treated in a state in which an image about the tissue is projected on the tissue. For example, the operation device includes at least one of a blood sampling device, a hemostatic device, a laparoscopic device including endoscopic and other instruments, an incisional device, and an abdominal operation device.

The surgery support system SYS includes a surgery lamp 80 and two display devices 31. The surgery lamp 80 includes a plurality of visible lighting lamps 81 that output visible light, a plurality of infrared LED modules 82, an infrared camera 71, and a projection unit 5. The infrared LED modules 82 are an irradiation unit that irradiates a tissue exposed in laparotomy with detection light. The infrared camera 71 is a light detection unit that detects light radiated from the tissue. The projection unit 5 can project an image generated by the control device (not shown) by using the detection result of the infrared camera (captured image). The display device 31 can display an image acquired by the infrared camera 71 and a component image generated by the control device. For example, a visible camera is provided to the surgery lamp 80, and the display device 31 can also display an image acquired by the visible camera.

The invasiveness and efficiency of an operation or treatment are determined by the range and intensity of injury or cautery associated with incision and hemostasis. The surgery support system SYS projects an image indicating information on a tissue on the tissue. Thus, a legion, as well as nerves, solid organs such as pancreas, fat tissue, blood vessels, and the like can be easily recognized to reduce invasiveness of an operation or treatment and enhance the efficiency of an operation or treatment.

The technical scope of the present invention is not limited to the above-described embodiments or modifications. For example, one or more elements described in the above-described embodiments or modifications may be omitted. The elements described in the above-described embodiments or modifications can be combined as appropriate.

### DESCRIPTION OF REFERENCE SIGNS

1 . . . scanning projection apparatus, 2 . . . irradiation unit, 3 . . . light detection unit, 4 . . . image generation unit, 5 . . . projection unit, 7 . . . projection optical system, 11 . . . imaging optical system, 15 . . . calculation unit, 16 . . . data generation unit, 22 . . . scanning unit, BT . . . tissue, SYS . . . surgery support system

What is claimed is:

1. A scanning projection apparatus comprising:
an irradiation unit that irradiates a biological tissue with detection light;
a light detection unit that detects light that is radiated from the tissue irradiated with the detection light;
an image generation unit that generates data on an image about the tissue by using a detection result of the light detection unit; and
a projection unit comprising a projection optical system that scans the tissue with visible light on the basis of the data, the projection unit being configured to project the image on the tissue through the scanning with the visible light.

2. The scanning projection apparatus of claim 1, wherein the projection optical system comprises a scanning unit that is capable of two-dimensionally scanning the tissue with the visible light.

3. The scanning projection apparatus of claim 1, wherein the image generation unit comprises:
a calculation unit that calculates information on components of the tissue by using a distribution of light intensity of the light detected by the light detection unit with respect to wavelength; and
a data generation unit that generates data on the image about the components by using a result calculated by the calculation unit.

4. The scanning projection apparatus of claim 3, wherein the calculation unit calculates information on the components about an amount of a first substance and an amount of a second substance included in the tissue by using a distribution of absorbance of the first substance with respect to wavelength and a distribution of absorbance of the second substance with respect to wavelength.

5. The scanning projection apparatus of claim 4, wherein the first substance is lipid and the second substance is water.

6. The scanning projection apparatus of claim 1, comprising a control unit that controls a wavelength of the detection light from the irradiation unit, wherein
the control unit outputs a first result that is detected by the light detection unit in a period during which the irradiation unit irradiates the tissue with light having a first wavelength and a second result that is detected by the light detection unit in a period during which the irradiation unit irradiates the tissue with light having a second wavelength separately to the image generation unit.

7. The scanning projection apparatus of claim 1, wherein
the light detection unit comprises a sensor having sensitivity to an infrared band of the detection light, and
an optical axis of the projection optical system on a light output side is set to be coaxial with an optical axis of the sensor on a light incident side.

8. The scanning projection apparatus of claim 1, comprising an imaging optical system that guides the light radiated from the tissue to the light detection unit, wherein
an optical axis of the imaging optical system and an optical axis of the projection optical system are set to be optically coaxial with each other.

9. The scanning projection apparatus of claim 1, wherein
the irradiation unit comprises a light source that outputs laser light as the detection light, and the projection optical system scans the tissue with the laser light, and
the light detection unit detects light that is radiated from the tissue irradiated with the laser light.

10. The scanning projection apparatus of claim 9, wherein a light source of the projection unit and the light source of the irradiation unit are disposed such that the visible light and the laser light pass through an optical path of the projection optical system.

11. The scanning projection apparatus of claim 1, wherein, in a period during which the projection unit displays the image for a first frame, the image generation unit generates data on the image for a second frame to be projected after the first frame.

12. The scanning projection apparatus of claim 1, wherein the projection unit is capable of adjusting at least one of color and brightness of the image.

13. The scanning projection apparatus of claim 1, comprising a casing in which the irradiation unit, the light detection unit, and the projection unit are provided.

14. A projection method comprising:
irradiating a biological tissue with detection light;
detecting, by a light detection unit, light that is radiated from the tissue irradiated with the detection light;
generating data on an image about the tissue by using a detection result of the light detection unit; and
scanning the tissue with visible light on the basis of the data, and projecting the image on the tissue through the scanning with the visible light.

15. The projection method of claim 14, comprising:
outputting laser light as the detection light;
scanning, by a projection optical system that scans the tissue with the visible light, the tissue with the laser light; and
detecting, by the light detection unit, light that is radiated from the tissue irradiated with the laser light.

16. A surgery support system comprising:
the scanning projection apparatus of claim 1; and
an operation device that is capable of treating the tissue in a state in which the image is projected on the tissue by the scanning projection apparatus.

17. A surgery support system comprising the scanning projection apparatus of claim 1.

18. A scanning apparatus comprising:
an irradiation unit that irradiates a target with detection light;
a detection unit that detects light that is radiated from the target irradiated with the detection light;
a generation unit that generates data on water or lipid in the target on the basis of a detection result of the detection unit; and
a scanning unit that scans the target with visible light on the basis of the data on water or lipid.

19. The scanning apparatus of claim 18, wherein the generation unit generates, as the data, an image indicating a distribution of water and a distribution of lipid in the target.

20. The scanning apparatus of claim 18, further comprising a control unit that switches between a mode of scanning the target with the visible light on the basis of the data on water and a mode of scanning the target with the visible light on the basis of the data on lipid.

21. The scanning apparatus of claim 18, wherein the scanning unit scans the target with the visible light in order to superimpose the image on at least a part of the target for display.

22. The scanning apparatus of claim 18, wherein the generation unit generates the data in which the water or the lipid is emphasized.

23. The scanning apparatus of claim 18, wherein the detection light has a wavelength based on absorbance of water and absorbance of lipid.

24. The scanning apparatus of claim 18, wherein the detection unit detects fluorescent light obtained by irradiating the target with the detection light including a wavelength of infrared light.

25. The scanning apparatus of claim 18, wherein the target comprises an affected area in a biological tissue.

26. A surgery support system comprising the scanning apparatus of claim 18.
